(19) United States
(12) Patent Application Publication
(10) Pub. No.: US 2018/0169224 A1
(43) Pub. Date: Jun. 21, 2018

(54) TLR AGONISTS FOR REDUCING ACTIVATION-INDUCED PD-1 EXPRESSION ON T CELLS AND METHODS OF USE

(71) Applicant: Wisconsin Alumni Research Foundation, Madison, WI (US)

(72) Inventors: Douglas G. McNeel, Madison, WI (US); Christopher D. Zahm, Madison, WI (US)

(21) Appl. No.: 15/838,824

(22) Filed: Dec. 12, 2017

Related U.S. Application Data
(60) Provisional application No. 62/432,999, filed on Dec. 12, 2016.

(51) Int. Cl. A61K 39/39; A61P 35/00; A61K 39/00

(52) U.S. Cl. CPC A61K 39/39; A61P 35/00; A61K 39/0011; A61K 2039/55511; A61K 2039/575; A61K 2039/585; A61K 2039/55516; A61K 2039/53

(57) ABSTRACT

The present invention is directed toward methods of increasing the immune response to an antigen using TLR1/2 agonist and/or TLR7 agonist in combination with a T cell activating treatment, for example, a vaccine. In some aspects, the present invention provides methods of enhancing an anti-tumor response comprising administering at least one TLR1/2 agonist and/or at least one TLR7 agonist in combination with an immunotherapeutic agent.

### CROSS-REFERENCE TO RELATED APPLICATIONS

This Application claims priority to U.S. Provisional Application No. 62/432,999 filed on Dec. 12, 2016, which is incorporated by reference in its entirety.

### STATEMENT REGARDING FEDERALLY SPONSORED RESEARCH

This invention was made with government support under W81XWH-15-1-0492 awarded by the ARMY/MRMC. The government has certain rights in the invention.

### BACKGROUND OF THE INVENTION

The field of the invention concerns improving the efficacy of immune responses to vaccines, particularly cancer vaccines. The invention also relates to new treatment for cancer by enhancing the immune response against cancer cells in combination with an immunotherapeutic agent.

Toll-like receptors (TLRs) are a class of proteins that play a key role in the innate immune system. TLRs are usually expressed in sentinel cells, such as macrophages and dendritic cells, that recognize structurally-conserved molecules derived from microbes. Microbes that have made it past epithelial barriers (skin, etc.) are recognized by TLRs, which activate immune cell responses. The TLR proteins include TLR1, TLR2, TLR3, TLR4, TLR5, TLR6, TLR7, TLR8, TLR9, TLR10, TLR11, TLR12, and TLR13, though the latter two are not found in humans.

Current anti-cancer DNA vaccines are safe and immunologically effective, but would be still more effective if made to give rise to a stronger anti-tumor response and to require fewer administrations. Thus, many recent studies have sought to identify methods for increasing the efficiency of plasmid DNA gene transfer and/or immunogenicity of the DNA. Current methods for increasing the immunogenicity of anti-cancer DNA vaccines include artificially increasing target antigen affinity for the T cell receptor, targeting high affinity neo-antigens, and other methods to increase the number of T cells stimulated/activated upon vaccination. However, some of these approaches have had limited success in increasing the immunogenicity in vivo and thus there is a need for other methods to increase the anti-tumor response.

Prostate cancer is a significant health risk for men over 50, with about 200,000 newly diagnosed cases each year in the United States (Jemal A. et al., Cancer Statistics, 2005 (2005) CA Cancer J Clin, 55:10-30). It is the most common tumor diagnosed among men and the second leading cause of male cancer-related death in the United States (Jemal et al., Cancer Statistics, 2003 (2003) CA Cancer J Clin, 53:5-26). Despite advances in screening and early detection, approximately 30% of patients undergoing definitive prostatectomy or ablative radiation therapy will have recurrent disease at 10 years (Oefelein et al., 1997, J Urol, 158:1460-1465). While immune checkpoint blockades have gained traction in treating many cancers, prostate cancer has been notoriously unaffected by this type of immunotherapeutic agent.

Therefore, the need to increase the immunogenicity of DNA vaccines by regulating the immune response and to increase the anti-tumor activity of the immune cells. Specifically, there is a need to overcome the low immunogenicity of DNA vaccines toward cancers, including methods for increasing the CD8+ T cell response to tumor antigens.

### SUMMARY OF THE INVENTION

The present invention overcomes the aforementioned drawbacks by providing improved responses to vaccines by administering TLR agonists, specifically TLR1/2 agonist or TLR7 agonists, in combination with an immunotherapeutic agent, for example, a vaccine.

In one aspect, a method of eliciting an anti-tumor response in a subject in need thereof is provided, the method comprising administering at least one TLR1/2 agonist or TLR7 agonist and an anti-tumor vaccine to the subject in an effective amount to elicit an anti-tumor response in said subject.

In another aspect, the disclosure provides a method of increasing the immune response to an antigen, the method comprising administering at least one TLR1/2 agonist or TLR7 agonist and at least one vaccine directed to said antigen in a subject, wherein the immune response to said antigen is increased.

In a further aspect, the present disclosure provides a method for increasing the efficacy of an immunotherapeutic agent, comprising the steps of administering at least one TLR1/2 agonist or TLR7 agonist and at least one immunotherapeutic agent to a subject in need thereof, wherein the at least one TLR1/2 agonist or TLR7 agonist is administered in an amount effective to increase the efficacy of the immunotherapeutic agent as compared to treatment with the immunotherapeutic agent alone.

In yet another aspect, the present disclosure provides kits for eliciting an anti-tumor response, the kit comprising: at least one TLR1/2 agonist or TLR7 agonist; and at least one cancer immunotherapeutic agent.

In another aspect, the present disclosure provides compositions for eliciting an anti-tumor response. In some aspects, the composition comprises at least one TLR1/2 agonist and/or at least one TLR7 agonist and at least one immunotherapeutic agent.

The foregoing and other aspects and advantages of the invention will appear in the following description. In the description, reference is made to the accompanying drawings which form a part hereof, and in which there is shown by way of illustration preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A. OT-1 transgenic splenocytes were treated with high affinity antigen in combination with Pam3CSK4 (TLR1 and 2 agonist) or Gardiquimod (TLR7 agonist) and levels of 4-1BB were used as a marker of T cell activation.

FIG. 1B. OT-1 transgenic splenocytes were treated with high affinity antigen in combination with Pam3CSK4 (TLR1 and 2 agonist) or Gardiquimod (TLR7 agonist) and PD-1 levels were assessed.

FIG. 1C. TLR2 or TLR7 agonist, when delivered with an anti-prostate tumor DNA vaccine in vivo, elicit greater anti-tumor responses. SSX2-expressing sarcoma tumor cells were implanted subcutaneously in HLA-A2 transgenic mice. On the day following implantation, and then weekly, mice were immunized intradermally with a DNA vaccine encoding SSX2 (pTVG-SSX2, blue, 4, 5, 6), SSX-2 opt (pTVG-SSX2-opt, red, 7, 8, 9), an optimized epitope of SSX2 with high affinity for HLA-A2 known to increase PD-1 expression on antigen-specific CD8+ T cells, or control vector (pTVG4, green, 1, 2, 3). Immunizations were given alone, or with Pam3CSK4 (TLR2 agonist) or Gardiquimod (TLR7 agonist). Tumor growth over time was monitored.

FIG. 2. Treatment of cells with a TLR agonist in combination with h T cell stimulation by an antigen affects PD-1 expression without affecting T cell activation. OT-1 transgenic splenocytes were stimulated in vitro for up to 96 hours with 2 μg/mL SIINFEKL peptide (SEQ ID NO:1) in the presence of TLR agonists (Pam3=Pam3CSK4, TLR1/2 agonist; PolyI:C=high molecular weight Poly(I:C), TLR3 agonist; MPLAs=Monophosphoryl Lipid A from Salmonella minnesota R595, TRL4 agonist; Gard=Gardiquimod, TLR7 agonist; R848=Resiquimod, TLR7/8 agonist; and ODN1826=CpG ODN, class B, TLR9 agonist). Cells were evaluated for T cell activation by 4-1BB expression (FIG. 2). Shown are the mean fluorescence intensity of cell surface receptor staining under each stimulation condition.

FIG. 3. Levels of PD-1 expression on T cells stimulated with antigen in the presence of TLR agonists as described in FIG. 2. Shown are the mean fluorescence intensity of cell surface receptor staining under each stimulation condition.

FIG. 4. Levels of LAG-3 expression in T cells activated with antigen in the presence of TLR agonists as described in FIG. 2. Shown are the mean fluorescence intensity of cell surface receptor staining under each stimulation condition.

FIG. 5. OT-1 transgenic splenocytes were treated with high affinity antigen for Ovalbumin (OVA) in combination with Pam3CSK4 (TLR1 and 2 agonist), Gardiquimod (TLR7 agonist), or no TLR agonist. The levels of various checkpoint markers or 4-1BB (evidencing T cell stimulation) were assayed

FIG. 6. OT-1 transgenic splenocytes were treated with high affinity antigen for OVA in combination with Pam3CSK4 (TLR1 and 2 agonist), Gardiquimod (TLR7 agonist) or no TLR agonist and the level of checkpoint marker BTLA was assayed.

FIG. 7. OT-1 transgenic splenocytes were treated with high affinity antigen for OVA in combination with Pam3CSK4 (TLR1 and 2 agonist), Gardiquimod (TLR7 agonist) or no TLR agonist and the level of checkpoint marker VISTA was assayed.

FIG. 8. OT-1 transgenic splenocytes were treated with high affinity antigen for OVA in combination with Pam3CSK4 (TLR1 and 2 agonist), Gardiquimod (TLR7 agonist) or no TLR agonist and the level of checkpoint marker CD160 was assayed.

FIG. 9. OT-1 transgenic splenocytes were treated with high affinity antigen for OVA in combination with Pam3CSK4 (TLR1 and 2 agonist), Gardiquimod (TLR7 agonist) or no TLR agonist and the levels of checkpoint marker CD244.2 was assayed.

FIG. 10. Proposed Model for PD-1 expression and its role in anti-tumor activity following various T cell activation conditions. In the model, if a high affinity epitope activates T cells (as in blue), PD-1 expression is highest and persistent. These T cells are least active in a PD-L1-expressing environment, as they are most able to be inactivated upon interacting with the PD-L1 receptor. When lower affinity epitopes activate T cells, shown in red, the cells still express PD-1, but this expression is not as high and/or does not persist. If

PD-L1 is prevented from binding the PD-1 expressed on the T cell during this time, those T cells retain anti-tumor activity. The T cells with potentially greatest anti-tumor activity, represented in green, are activated T cells for which PD-1 expression or signaling is blocked at the time of T cell activation. The arrow represents the time of T cell activation by the T cell specific epitope.

FIG. 11. Vaccine type can affect expression of T-cell checkpoint molecules on activated CD8+ T cells. OT-1 transgenic T cells were adoptively transferred into wild-type B6 mice. Mice were immunized intradermally with encoding vector containing a polynucleotide that encodes ovalbumin (OVA DNA) or with the vector lacking the ovalbumin-encoding polynucleotide as a control (DNA vector), intraperitoneally with Listeria monocytogenes (Lm) containing a vector that encodes ovalbumin (Lm-OVA)or Lm containing empty vector as a control (Empty Lm), subcutaneously with the SIINFEKL (SEQ ID NO:1) peptide (OVA peptide) or non-specific peptide (NS peptide), or with the indicated combinations of the above. Two days later splenocytes were collected and the phenotype of OT-1 T cells was assessed via flow cytometry. Shown are the mean fluorescence intensity (MFI) of 4-1BB, PD-1 and LAG-3 surface expression on p103-specific tetramer+ CD8+ CD44+ T cells. Individual data points are from 5 mice per group, with mean and standard error shown. Asterisk indicates P<0.05.

FIGS. 12A-12C. TLR stimulation at the time of T-cell activation in vitro affects expression of PD-1. Splenocytes were prepared from the spleens of OT-1 mice and stimulated in vitro with the high-affinity SIINFEKL (SEQ ID NO:1) (A), the moderate-affinity SIINTEKL (SEQ ID NO:2) (B), or the low affinity SIINFEKP (SEQ ID NO:7) (C) peptides in the presence or absence of specific TLR agonists (TRL1/2 (Pam3CSK4), TRL3 (Poly I:C), TRL4 (MPLAs), TRL7 (Gardiquimod), TRL7/8 (R848), or TRL9 (ODN1826)). Shown are the MFI of 4-1BB, PD-1 and LAG-3 on cells collected daily for 4 days. Asterisks indicate P<0.01, with each comparison made to stimulation with the SIINFEKL (SEQ ID NO:1) peptide alone, and color corresponding to the agent compared. Results are from one experiment, with samples assessed in triplicate, and are representative of four similar experiments.

FIG. 13. The effect of TLR1/2, TLR7 and TLR9 agonists is greatest on PD-1 following T-cell activation. OT-1 splenocytes were stimulated with either the OVA peptide (SIINFEKL, SEQ ID NO:1) or a non-specific peptide control (NS), alone or in combination with the designated TLR agonists. At daily intervals up to 4 days, the cell surface expression of multiple immune checkpoints (PD-1, LAG3, CTLA-4, TIM3, VISTA, CD244.2, TIGIT, and CD160) and 4-1BB was assessed by flow cytometry. Asterisks indicate P<0.01, with each comparison made to stimulation with peptide alone, and color corresponding to the treatment group compared. Results are from one experiment, with samples assessed in triplicate, and are representative of two similar experiments.

FIGS. 14A-14D. Changes in PD-1 expression following TLR stimulation are mediated by IL-12 secreted from APCs. Panel A: Purified OT-1 T cells (T), B cells (B), or dendritic cells (DC) were activated with SIINFEKL peptide (SEQ ID NO:1, OVA) or with a non-specific peptide (NS) in the presence of either TLR1/2 (TLR1/2) agonist, TLR7 agonist (TLR7), or media alone. After 24 hours, cells were washed and co-cultured with naïve purified OT-1 CD8+ T cells for an additional 24 hours and then assessed for expression of the indicated surface receptors (4-1BB, CD3, PD-1, LAG3, CTLA-4, and TIM3) by flow cytometry. Shown is the MFI for each marker, with samples assessed in triplicate. Panel B: Purified DC were stimulated in the presence of TLR1/2 agonist, TLR7 agonist, or media alone. After 24 hours, media was collected and used for culture of purified OT-1 CD8+ T cells with naïve DC in the presence of SIINFEKL peptide (SEQ ID NO:1, OVA) or non-specific (NS) peptide. After 24 hours, cells were assessed for expression of the indicated surface receptors (4-1BB, PD-1, LAG3, and CTLA-4) by flow cytometry. Shown is mean and standard error of the MFI for each marker, with samples assessed in triplicate. Panel C: Purified DC were stimulated in the presence of TLR1/2 agonist (Pam3), TLR3 agonist (PolyIC), TLR4 agonist (MPLA), TLR7/8 agonists (Gard, R848), TLR9 agonist (ODN), or media only (none) for 24 hours. Culture supernatant was then evaluated for the presence of IL12 p70 by ELISA. Asterisks indicate significant (p<0.05) IL-12 release compared to media alone. Panel D: Purified DC were stimulated in the presence of TLR1/2 agonist, TLR7 agonist, or media alone. After 24 hours, medium was collected and used for culture of purified OT-1 CD8+ T cells with naïve DC in the presence of SIINFEKL peptide (SEQ ID NO:1, OVA) and, where indicated, 0.1 ng/mL IL-12 or 0.12 μg/mL anti-IL-12 antibody. After 24 hours, cells were assessed for expression of the indicated surface receptors (4-1BB, PD-1, LAG3, and CTLA-4) by flow cytometry. Shown is mean and standard error of the MFI for each marker, with samples assessed in triplicate and are representative of two similar experiments. Asterisks indicate P<0.01.

FIGS. 15A-15B. T-cells activated in the presence of TLR1/2 or TLR7 ligands exhibit a gene expression profile consistent with enhanced effector function. OT-1 splenocytes were activated for 24 or 72 hours in the presence of SIINFEKL (SEQ ID NO:1) peptide alone, or with TLR1/2 or 7 ligands. CD8+ OT-1 T cells were then purified and evaluated for gene expression changes by RNAseq. Panel A: Venn diagrams showing the number of enriched GO-term expression with significantly increased (up) or decreased (down) expression following treatment with TLR1/2 (TLR2) or TLR7 relative to SIINFEKL (SEQ ID NO:1) treatment alone. Results are from Gene Set Enrichment Analysis (GSEA) with a false discovery rate (FDR, estimated probability that a gene set with a given NES represents a false positive finding) of <0.25. Panel B: Shown are gene expression changes, in the presence of TLR agonist and OVA peptide (SEQ ID NO:1) versus OVA peptide alone, for individual genes associated with different T cell effector functions (green=increased expression, red=reduced expression).

FIG. 16. TLR stimulation at the time of T-cell activation reduces expression of PD-1 in vivo. OT-1 T cells were adoptively transferred to naïve C57BL/6 mice and immunized once with 100 μg OVA peptide (SEQ ID NO:1) or OVA FT peptide (SEQ ID NO:2) or non-specific peptide (NS) one day later in the presence or absence of TRL1/2, TLR7, or TLR9 ligands. Splenocytes were collected from groups up to 14 days after immunization and evaluated for expression of 4-1BB, PD-1 and LAG-3 by flow cytometry. Shown are the MFI of 4-1BB, PD-1 and LAG-3 on OT-1 CD8+ T cells collected. Asterisks indicate P<0.01, with each comparison made to peptide alone, and color corresponding to the treatment group compared. Results are from one experiment, with N=5 mice/group, and are representative of two similar experiments.

FIGS. 17A-17C. Peptide immunization in the presence of TLR1/2 or TLR7 ligands, co-delivered as vaccine adjuvants, elicits greater anti-tumor immunity in vivo: Ovalbumin-expressing E.G7 cells were implanted in C57BL/6 mice and permitted to grow until palpable (-14 days). OT-1 T cells were then adoptively transferred and mice were immunized the following day with SIINFEKL (SEQ ID NO:1, OVA peptide) or FT peptide alone, with TLR1/2 or TLR7 agonist, or in combination with 100 μg anti-PD-1 antibody delivered the day following immunization. Panel A: Shown are the tumor growth curves (median+−standard error, n=5 mice per group). Results are from one experiment and are representative of two similar experiments. Panel B: Tumors obtained at necropsy were evaluated for the % of infiltrating CD8+ T cells among CD45+ cells (upper panel), and expression of PD-L1 on CD45-cells (lower panel). Panel C: Tumor-infiltrating CD8+ T cells were evaluated for 4-1BB, PD-1 and LAG-3 expression by flow cytometry. Asterisks indicates P<0.05.

FIG. 18. DNA immunization in the presence of TLR1/2 or TLR7 ligands elicits greater anti-tumor efficacy in vivo. HLA-A2-expressing transgenic mice were implanted with SSX-2-expressing tumor cells. After 2 days mice were immunized weekly with control vector (pTVG4), DNA encoding native SSX2 (pTVG-SSX2), or DNA encoding high affinity HLA-A2 epitopes (pTVG-SSX2 opt) in the presence or absence of TLR1/2 or TLR7 agonist. Shown are the growth curves for each group (n=6 animals per group). Asterisk indicates P<0.05. Results are representative of two independent experiments with 12 total mice per group.

FIGS. 19A-19C. TLR stimulation at the time of T-cell activation with lower affinity epitopes in vitro affects expression of PD-1. OT-1 splenocytes were stimulated with either the moderate affinity SIINTEKL (SEQ ID NO:2) (A), the lower affinity SIINFEKP (SEQ ID NO:7) (B), or the low affinity SIINFEKY (SEQ ID NO:8) (C) peptides, alone or in combination with the designated TLR agonists. At daily intervals up to 4 days, expression of multiple immune checkpoints (PD-1, LAG3, CTLA-4, TIM3, VISTA, CD244.2, TIGIT, and CD160) and 4-1BB was assessed by flow cytometry. Asterisks indicate: P<0.01. Results are from one experiment, with samples assessed in triplicate, and are representative of two similar experiments.

FIG. 20. TLR stimulation at the time of T-cell activation in vitro affects expression of PD-1 that is not affected by blockade of CD80, CD86, or OX40L. Splenocytes were prepared from the spleens of OT-1 mice and stimulated in vitro with the SIINFEKL (SEQ ID NO:1, OVA) peptide in the presence or absence of specific TLR agonists (TRL1/2 (Pam3CSK4) or TRL9 (ODN1826), or blocking antibodies for OX40L (0.05 μg/mL), CD80 (0.6 μg/mL), or CD86 (5 μg/mL). Shown is the MFI of PD-1 on CD3+CD4−CD8+ T-cells collected daily for 4 days. Asterisks indicate: P<0.01. Results are from one experiment, with samples assessed in triplicate, and are representative of two similar experiments.

FIGS. 21A-21B. Changes in PD-1 expression following TLR stimulation are mediated by IL-12 secreted from APCs. Panel A: Purified B cells were stimulated in the presence of TLR1/2 agonist, TLR7 agonist, or media alone. After 24 hours, media was collected and used for culture of purified OT-1 CD8+ T cells with naïve B cells in the presence of SIINFEKL (SEQ ID NO:1, OVA) or non-specific (NS) peptide. After 24 hours, cells were assessed for expression of the indicated surface receptors (4-1BB, PD-1, LAG-3, and CTLA-4) by flow cytometry. Shown is mean and standard deviation of the MFI for each marker, with samples assessed in triplicate. Panel B: Purified B cells were stimulated in the presence of TLR1/2 agonist, TLR7 agonist, or media alone. After 24 hours, medium was collected and used for culture of purified OT-1 CD8+ T cells with naïve DC cells in the presence of SIINFEKL peptide (SEQ ID NO:1, OVA) and, where indicated, 0.1 ng/mL IL-12 or 0.12 μg/mL anti-IL-12 antibody. After 24 hours, cells were assessed for expression of the indicated surface receptors (4-1BB, PD-1, LAGS, and CTLA-4) by flow cytometry. Asterisks indicate: P<0.01. Shown is mean and standard error of the MFI for each marker, with samples assessed in triplicate and are representative of two similar experiments.

FIG. 22. TLR1/2 or TLR7 ligand adjuvants alone do not have significant anti-tumor efficacy in the absence of antigen-specific T-cell activation: Ovalbumin-expressing E.G7 cells were implanted in C57BL/6 mice and permitted to grow until palpable (-14 days). OT-1 T cells were then adoptively transferred and mice were immunized the following day with the OVA peptide (SIINFEKL, SEQ ID NO:1) or non-specific (NS) peptide, alone or with TLR1/2 (Pam3) or TLR7 (Gard) agonist. Shown are the tumor growth curves (median+/−standard error, n=5 animals per group). Asterisks indicate P<0.001 Results are from one experiment, with N=6 mice/group.

### DETAILED DESCRIPTION OF THE INVENTION

The present invention has been described in terms of one or more preferred embodiments, and it should be appreciated that many equivalents, alternatives, variations, and modifications other than those expressly stated are also possible and within the scope of the invention.

The present disclosure is directed to the surprising finding that agonists of specific TLRs, specifically TLR1/2 and TLR7, are able to increase the anti-antigen response, specifically the anti-tumor response, of activated immune cells (e.g., CD8+ T cells). This increased anti-tumor response reduces and inhibits tumor growth, and, in some cases, tumor ablation, in an in vivo cancer model. The inventors have found that when an immunotherapeutic agent such as a vaccine is administered in combination with a TLR1/2 agonist and/or a TLR7 agonist, antigen-specific immune cells are activated and PD-1 expression in these activated immune cells is down-regulated and/or inhibited such that immune response to the antigen is enhanced. This result is specific to certain TLR agonists (e.g., TLR1/2 agonist and TRL7 agonist) and is not applicable to all TLR agonists. As shown in Table 1, tumors completely regressed in half of the mice treated with a DNA vaccine in combination with a TLR1/2 agonist or TLR7 agonist.

Immunization using an anti-tumor DNA vaccine increases expression in tumor cells of PD-L1, which is associated with subsequent development of tumor antigen-specific IFNγ secretion, in both animals and human patients. Without being bound by any theory, it is thought that, at least in some instances, increased PD-L1 expression inactivates activated and trafficked immune cells, and down-regulates cell-mediated immune responses. In other words, it is believed that when these high PD-1-expressing T cells reach tumor cells decorated with PD-L1, these T cells are more likely to be deactivated than T cells expressing lower levels of PD-1, such that the cytotoxic immune response against the tumor cells is decreased. Such T cells include T cells that are activated by an enhanced antigen (i.e. antigen that have been modified to increase their affinity for a TCR) as these T cells express higher levels of PD-1.

The anti-tumor response could be restored if vaccination was combined with PD-1/PD-L1 blockade. Id. However, anti-PD-1 treatment has had limited success in vivo. The inventors have shown that blocking the PD-1 pathway would beneficially improve immunogenicity of cancer vaccines and have also surprisingly found that TLR1/2 agonists and TLR7 agonists can reduce expression of PD-1 on activated immune cells and increase immune response to the target antigen.

The inventors here show that TLR agonists reduce PD-1 expression in T cells and unexpectedly improve vaccine efficacy by increasing the immune response to the target antigen. The present disclosure provides methods for improving efficacy of vaccines, including cancer vaccines, and for improving anti-tumor responses, by administering TLR agonists, specifically TLR1/2 agonist and/or TLR7 agonists in combination with an immunotherapeutic agent. Data obtained in a mouse model system demonstrates that pre-dosing a tumor with either a TLR1/2 agonist (e.g., Pam3CSK4) or TLR7 agonist (Gardiquimod) improved the tumor growth suppression after a DNA cancer vaccine was given to mice. Stimulation with TLR agonists led to an almost complete tumor growth suppression, significantly better than the effect seen in animals given the vaccine alone. These results are specific to TLR1/2 agonist and TLR7 agonist, as other TLR agonists do not have such an effect on PD-1 expression or anti-tumor responses as demonstrated in the examples below.

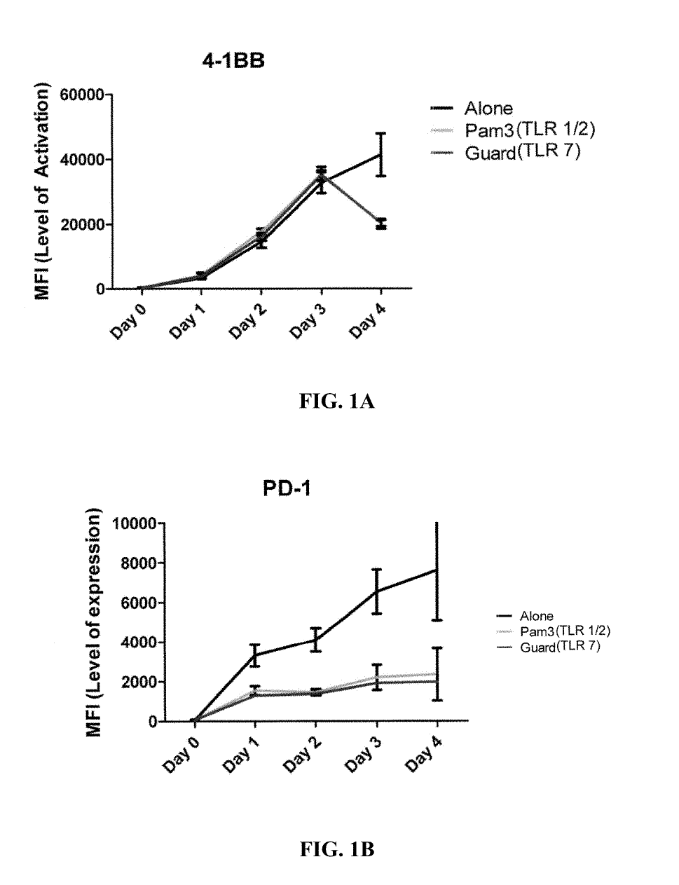

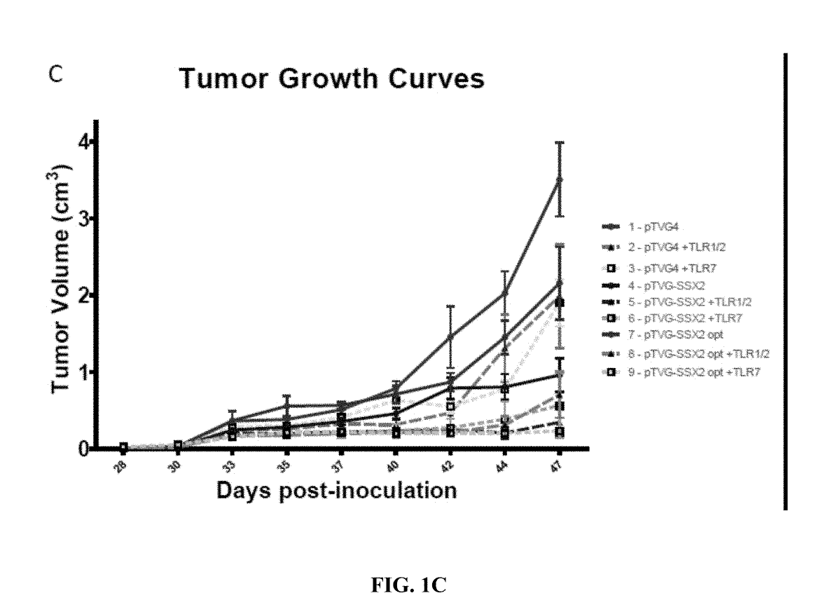

Including TLR agonists during stimulation of the immune system by a vaccine can ameliorate the anti-vaccine effect, resulting in greater T cell activation without increasing expression of regulatory receptors, including PD-1 (FIGS. 1A, 1B). Using mouse models, the inventors demonstrated that this combination results in greater anti-tumor efficacy when compared to the vaccine or agonists alone (e.g., FIGS. 1C, 17 and 18). Thus treatment with TLR agonists combined with strategies to increase tumor-specific CD8+ T cells results in an increase in activated and unregulated tumor-specific T cells that are less susceptible to immune checkpoint mediated regulation, opening up the possibility for more effective cancer treatments.

The methods provided herein comprise administering at least one TLR1/2 agonist and/or at least one TLR7 agonist in combination with at least one immunotherapeutic agent to a subject in order to improve the efficacy of the immunotherapeutic agent, improve, enhance and/or increase the immune response against a specific antigen or immunogen, and/or increase the anti-tumor response in a subject. In some embodiments, a combination of at least one TLR1/2 agonist and at least one TLR7 agonist are both administered to the subject.

In some embodiments, the disclosure provides a method of increasing the immune response to an antigen, the method comprising administering at least one TLR1/2 or TLR7 agonist and at least one immunotherapeutic agent (e.g., vaccine) directed to said antigen in a subject, wherein the immune response to said antigen is increased. In some embodiments, the immune response is a cellular immune response, specifically a CD8+ T cell response. In some embodiments, the immune cells exhibit a PD-1^{low} phenotype.

As used herein, the term PD-1^{low} phenotype refers to the level of expression of PD-1 on the surface of the immune cells (e.g., CD8+ T cells). Specifically, PD-1^{low} phenotype refers to the level of PD-1 expression on the immune cells, as being low including no expression as determined by flow cytometry and fluorescence staining. In some embodiments, a majority of the activated immune cells are PD-1^{low}, where the majority is at least 55%, alternatively at least 65%, alternatively at least 70%, alternatively at least 75%, alternatively at least 80%, alternatively at least 90%, alternatively at least 95% PD1^{low}, including any percentages in-between (e.g., at least 56%, 57%, 58%, 59%, 60%, 61%, 62%, 63%, 64%, 65%, 66%, 67%, 68%, 69%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%). In a preferred embodiment, the majority is at least 85%, preferably at least 90%, PD-1^{low}.

An “effective treatment” refers to treatment producing a beneficial effect, e.g., amelioration of at least one symptom of a disease or cancer. A beneficial effect can take the form of an improvement over baseline, i.e., an improvement over a measurement or observation made prior to initiation of therapy according to the method. A beneficial effect can also take the form of reducing, inhibiting or preventing further growth of cancer cells, reducing, inhibiting or preventing metastasis of the cancer cells or invasiveness of the cancer cells or metastasis or reducing, alleviating, inhibiting or preventing one or more symptoms of the cancer or metastasis thereof. Such effective treatment may, e.g., reduce patient pain, reduce the size or number of cancer cells, reduce or prevent metastasis of a cancer cell, or may slow cancer or metastatic cell growth.

The terms “effective amount” or “therapeutically effective amount” refer to an amount sufficient to effect beneficial or desirable biological or clinical results. That result can be reducing, alleviating, inhibiting or preventing one or more symptoms of a disease or condition, reducing, inhibiting or preventing the growth of cancer cells, reducing, inhibiting or preventing metastasis of the cancer cells or invasiveness of the cancer cells or metastasis, or reducing, alleviating, inhibiting or preventing one or more symptoms of the cancer or metastasis thereof, or any other desired alteration of a biological system. In some embodiments, the effective amount is an amount suitable to provide the desired effect, e.g., anti-antigen or anti-tumor response.

The terms “metastasis” or “secondary tumor” refer to cancer cells that have spread to a secondary site, e.g., outside of the original primary cancer site. Secondary sites include, but are not limited to, for example, the lymphatic system, skin, distant organs (e.g., liver, stomach, pancreas, brain, etc.) and the like and will differ depending on the site of the primary tumor.

The terms “subject” and “patient” are used interchangeably and refer to any animal (e.g., a mammal), including, but not limited to, humans, non-human primates, rodents, and the like, which is to be the recipient of a particular treatment. Typically, the terms “subject” and “patient” are used interchangeably herein in reference to a mammalian, for example, human subject. For example, a suitable subject includes a subject in need of cancer treatment.

The term “CD8+ T cell response” refers to the activation of CD8+ cells to kill cells expressing the antigen to which the T cell was activated. CD8+ T cells may kill the cells through different mechanisms, including, the secretion of cytokines, primarily TNF-α and IFN-γ, which have anti-tumor and anti-viral microbial effects, the production and release of cytotoxic granules, e.g., perforin, and granzymes, and/or destruction of infected cells is via Fas/FasL interactions.

In some embodiments, the methods increase the anti-tumor response. Such increase in the anti-tumor response may be demonstrated by an increased anti-tumor response in an animal model of the tumor as compared with the animal model without treatment or with administration of the T cell activating agent alone. For example, an increase anti-tumor response may be observed after the administration of the at least one TRL1/2 agonist and/or TLR7 agonist in combination with a T cell activating agent, e.g., an immunotherapeutic agent (e.g. vaccine) and demonstrated by a measurable increase in the anti-tumor response. The anti-tumor response may be demonstrated by a decrease in tumor size and volume within the animal or an increase in CD8+ T cell activation as compared to treatment with the DNA vaccine alone or as compared to the untreated control.

The methods disclosed herein can include a conventional treatment regimen, which can be altered to include the steps of the methods described herein. The methods disclosed herein can include monitoring the patient to determine efficacy of treatment and further modifying the treatment in response to the monitoring.

TLR1/2 agonists and TLR7 agonists are known in the art and used herein include, but are not limited to, for example, Pam3Cys (a TLR1/2 agonist), CFA (a TLR-2 agonist), MALP2 (a TLR-2 agonist), Pam2Cys (a TLR-2 agonist), FSL-1 (a TLR-2 agonist), Hib-OMPC (a TLR-2 agonist), imiquimod (a TLR-7 agonist), resiquimod (a TLR-7/8 agonist), loxoribine (a TLR-7/8 agonist), guardiquimod (TLR-7 agonist) and the like. Further examples of suitable TLR2 agonists include TLR-2 ligands, including, for example, lipoglycans including lipoarabinomannan (e.g. from M smegmatis) and lipomannan (e.g. from M. smegmatis), lipopolysaccharides (LPS) for example LPS from P. gingivalis (LPS-PG), lipoteichoic acids (LTA, e.g., from LTA from B. substilis or S. aureus), peptidocylcans PGN, for example PGN from Bacillus subtilis, PGN from E. coli 0111:B4, PGN from Escherichia coli K12, PGN from Staphylococcus aureus, synthetic lipoproproteins (e.g. CL429, FLS-1, Pam2CSK4, Pam3CSK4), Zymosan and the like. Further examples of suitable TLR7 agonist include, for example, thiazoquinoline compounds (e.g. CL075), imidazoquinoline compounds (e.g., CL097, Gardiquimod™, Imiquimod (R837), R848, etc.), CL264 (adenine analog), CL307, Loxorbine (guanosine analog), Poly(dT), TL8-506 and the like.

In one embodiment, the present disclosure provides a method of eliciting, enhancing or improving an anti-tumor response in a subject in need thereof. The method comprises administering at least one TLR1/2 and/or TLR7 agonist and an immunotherapeutic agent (for example, an anti-tumor vaccine) to the subject in an effective amount to elicit an anti-tumor response in said subject. In some embodiments, the anti-tumor response is improved over the response to the immunotherapeutic agent or vaccine alone. In some embodiments, the anti-tumor response is a cell-mediated immune response. In some embodiments, the activated T cells, e.g., CD8+ T cells, express low levels of PD-1 or do not express PD-1. In other words, the activated T cells have a PD-1^{low} phenotype. Suitably, the subject is a human subject suffering from cancer, preferably prostate cancer.

An “improved immune-mediated anti-tumor response” means an increase in the ability of one or more immune cells to recognize tumor cells. In some instances, the improved immune-mediated anti-tumor response results in an increased ability of one or more immune cells to target/recognize and kill cancer cells (e.g., CD8+ T cells). An improved immune-mediated anti-tumor response may be seen as a reduction in the number of cancer cells, inhibiting, retarding or slowing the growth of cancer cells, inhibiting, retarding or slowing the metastasis of cancer cells, increased infiltration of cytotoxic T cells into the tumor, or decreased inhibition of immune population within the tumor microenvironment.

The terms “cell-mediated immune response” or “cell-mediated immunity” refer to an immune response mediated by immune cells and does not involve antibodies (humoral immune response). Specifically, cell-mediated immune response includes antigen-specific cytotoxic T-lymphocytes (CD8+ T cells) or activation of phagocytes. Phagocytes include white blood cells such as neutrophils, monocytes, macrophages, mast cells, and dendritic cells. In a preferred embodiment, the cell-mediated immune response is a cytotoxic T cell response or CD8+ T cell response.

The disease, disorder or condition that can be treated by the methods of the present invention include a viral disease, a bacterial disease, a parasitic disease, a cancer, an autoimmune disease, an allergy or other disease. In a preferred embodiment, the disease is cancer, more specifically prostate cancer.

The term “immunotherapeutic agent” refers to an agent that results in or elicits an immune response for the treatment of a disease, condition or disorder. An immunotherapeutic agent is an agent used to treat a disease by inducing, amplifying or enhancing an immune response against antigens specific to the disease, for example, antigens expressed on cancer cells. In some instances, immunotherapeutic agent may be a cell-based immunotherapeutic agent that employs target immune effector cells such as lymphocytes, macrophages, dendritic cells, natural killer cells (NK cells), cytotoxic T lymphocytes (CTL), and the like to target abnormal antigens expressed on the surface of cancer/tumor cells. In a preferred embodiment, at least one immunotherapeutic agent may be a T cell immunotherapeutic agent. Suitable T cell immunotherapies are known in the art and include, but are not limited to, for example, vaccines (e.g., DNA vaccine), oncolytic viral therapies that engage/recruit T cells, adoptive immunotherapies approaches (e.g., chimeric antigen receptor (CAR) T cells), or bispecific T cell engagers (BiTEs). CAR is transmembrane protein containing an extracellular portion containing a recognition or binding site for the strong antigen and a transmembrane and intracellular domain capable of signal transduction to activate the lymphocyte (e.g., T cell). CAR are known in the art and can be made using standard techniques. Suitable vaccines include vaccines that result in the stimulation of effector cells. Suitable vaccines include, but are not limited to, for example, peptides, genetically modified viruses or viral particles, and tumor cell lysates. In some embodiments, the vaccine can be combined with the adoptive transfer of T cells (e.g., Adoptive Cell Therapy or ACT) specific for tumor antigens or the transfer of CAR T cells that are specific for a tumor antigen.

The term “vaccine,” as used herein, refers to a biological preparation that contains antigen or immunogen that can elicit an immune response. The antigen or immunogen can be, for example, an infectious agent, a molecule that resembles a disease-causing microorganism or cell, or a protein associated with an abnormal or diseased cell (e.g., tumor associated antigen). For example, antigens or immunogens may be made from an attenuated or inactivated form of said microorganism or cell or its toxins. A vaccine is administered to an individual in order to stimulate that individual's immune response to said antigen or immunogen.

In some embodiments, the antigen is a tumor antigen or an infectious agent. In a preferred embodiment, the antigen is a tumor-associated antigen. A tumor antigen or tumor-associated antigen is an antigen that is preferentially expressed on the surface of a tumor cell and not expressed on normal, healthy cells.

Suitable infectious agents include, but are not limited to, for example, a virus, a bacteria, a fungus, a parasite, and the like. Suitable viruses include, but are not limited to, adenoviruses, herpesviruses, poxviruses, parvoviruses, reoviruses, picornaviruses, togaviruses, orthomyxoviruses, rhabdoviruses, retroviruses, hepadnaviruses, herpesviruses, rhinoviruses, including, but not limited to, for example, Epstein-Barr viruses (EBV), cytomegalovirus, Karposi sarcoma virus, human papillomavirus (HPV), human immunodeficiency virus (HIV), herpes simplex virus, herpesvirus 1, herpesvirus 2, herpesvirus 6, herpesvirus 7, herpesvirus 8, hepatitis A, hepatitis B, hepatitis C, measles, mumps, parvovirus, rabies virus, rubella virus, influenza virus, varicella zoster virus, ebola virus, west niles virus, yellow fever virus, dengue virus, rotovirus, zika virus, and the like. Suitable bacteria include, but are not limited to, for example, Escherichia coli, Salmonella, Helicobacter pylori, Neisseria gonorrhoeae, Neisseria meningitides, Streptococcus, bacillus, tuberculosis, leprosy, Legionella, Listeria and Brucella and the like. Suitable parasites include, but are not limited to, for example, parasites malaria, Leishmania, Cryptosporidium, Cyclospora, Toxoplasma gondii, Plasmodium spp and the like.

The term “antigen,” as used herein, refers to any molecule that is recognized by the immune system and that can stimulate an immune response.

The term “immunogen,” as used herein, refers to any molecule that is recognized by the immune system and is able to induce an immune response, preferably a cell-mediated immune response (e.g., CD8+ T cell response).

The term “cancer” and “tumor” as used herein interchangeably and are meant to encompass any cancer, neoplastic and preneoplastic disease that is characterized by abnormal growth of cells. Cancer may, for example, be selected from the group consisting of colon carcinoma, breast cancer, pancreatic cancer, ovarian cancer, prostate cancer, head and neck cancer, lung cancer, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, fibrosarcoma, myxosarcoma, liposarcoma, chondrosarcoma, osteogenic sarcoma, chordoma, angiosarcoma, endotheliosarcoma, Ewing's sarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, endometrial cancer, lung carcinoma, small cell lung carcinoma, bladder carcinoma, epithelial carcinoma, glioblastomas, neuronomas, craniopharingiomas, schwannomas, glioma, astrocytoma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, childhood-null acute lymphoid leukemia (ALL), thymic ALL, B-cell ALL, acute megakaryocytic leukemia, Burkitt's lymphoma, and T cell leukemia, small and large non-small cell lung carcinoma, acute granulocytic leukemia, germ cell tumors, endometrial cancer, gastric cancer, hairy cell leukemia, thyroid cancer and other cancers known in the art. In a preferred embodiment, the cancer is prostate cancer.

In a particular embodiment, the subject may suffer from prostate cancer. The immunotherapeutic agent may be a DNA vaccine targeted to prostate cancer. Suitable vaccines are known in the art and include, for example, a recombinant DNA vaccine that encodes the androgen receptor or fragments thereof or a peptide vaccine comprising a polypeptide androgen receptor or fragments thereof. Suitable recombinant DNA vaccines are disclosed in U.S. Pat. No. 7,910,565, 8,513,210 and 8,962,590, entitled “Prostate cancer vaccine,” and U.S. Pat. No. 7,179,797 and U.S. application Ser. No. 11/615,778 entitled “Methods and compositions for treating prostate cancer using DNA vaccines,” which are incorporated by reference in their entireties. In some embodiments, the DNA vaccine comprises pTVG-AR (pTVG-AR or pTVG-ARLBD refer to the same vector and both designations are used interchangeably herein). pTVG-AR is a vector comprising the coding sequence for the ligand-binding domain of the human androgen receptor gene inserted into the pTVG4 vector to create the immunization vector pTVG-AR, as disclosed in U.S. Pat. No. 7,910,565, incorporated by reference in its entirety. According to another embodiment, the DNA vaccine comprises a polynucleotide operatively linked to a transcriptional regulatory element (e.g., a promoter such as a heterologous promoter) wherein the polynucleotide encodes a member selected from (i) a mammalian androgen receptor (e.g., a human androgen receptor), (ii) a fragment of the androgen receptor that comprises the ligand-binding domain, (iii) a fragment of the ligand-binding domain defined by SEQ ID NO:3(LLLFSIIPV); (iv) a fragment of the ligand-binding domain defined by SEQ ID NO:4 (RMLYFAPDLV), (v) a fragment of the ligand-binding domain defined by SEQ ID NO:5 (FLCMKALLL), and (vi) a fragment of the ligand-binding domain defined by SEQ ID NO:6 (QLTKLLDSV), wherein administration of said vaccine to a subject induces a cytotoxic immune reaction against cells expressing androgen receptor. Other suitable DNA vaccines encode native or modified SSX2 as described in Smith et al. 2011 (Vaccines targeting the cancer-testis antigen SSX-2 elicit HLA-A2 epitopes specific cytolytic T cells. J. Immunother 2011:34:569-80) and Smith et al. 2014 (DNA vaccines encoding altered peptide ligands for SSX2 enhance epitope-specific CD8+ T cell immune responses. Vaccine 2014:32:1707-15), the contents of which are incorporated by reference in their entireties. Another suitable vaccine for prostate cancer is a DNA vaccine encoding prostatic acid phosphatase (PAP), which is disclosed in U.S. Pat. No. 7,179,797, the contents of which are incorporated in its entirety.

Androgen receptor genes are known and have been cloned from many species. For example, the human, mouse, rat, dog, chimpanzee, macaque, and lemur androgen receptor cDNA along with amino acid sequences can be found at GenBank Accession Nos. NM_000044 (SEQ ID NO:9), NM_013476 (SEQ ID NO:10), NM_012502 (SEQ ID NO:11), NM_001003053 (SEQ ID NO:12), NM_001009012 (SEQ ID NO:13), U94179 (SEQ ID NO:14), and U94178 (SEQ ID NO:15), respectively, which are incorporated by reference in their entirety.

In some embodiments, the method of eliciting, increasing or enhancing an anti-tumor response comprises administering a TLR1/2 agonist in combination with a TLR7 agonist and at least one immunotherapeutic agent. In a preferred embodiment, the immunotherapeutic agent is an anti-tumor vaccine, preferably a DNA vaccine.

In another embodiment, the disclosure provides a method of eliciting, increasing or enhancing the immune response to an antigen, the method comprising administering at least one TLR1/2 or TLR7 agonist and at least one immunotherapeutic agent directed to said antigen in a subject, wherein the immune response to said antigen is increased relative to a subject treated with the immunotherapeutic agent alone. In a preferred embodiment, the immune response is a cellular immune response, preferably a CD8+ T cell response. In a preferred embodiment, the immunotherapeutic agent is a vaccine. The method results in the activation of CD8+ T cells which exhibit a PD-1^{low} phenotype.

In a further embodiment, a method for increasing the efficacy of an immunotherapeutic agent is provided. The method comprises the steps of administering at least one TLR1/2 and/or TLR7 agonist and at least one immunotherapeutic agent to a subject in need thereof, wherein at least one TLR1/2 and/or TLR7 agonist is administered in an amount effective to increase the efficacy of the immunotherapeutic agent. In some preferred embodiments, the immunotherapeutic agent is a DNA vaccine. In some aspects, the administration of the at least one TLR1/2 and/or TLR7 agonist results in an increase in activation of CD8+ T cells in the subject. Suitably, this increased activation of CD8+ T cells is specific to CD8+ T cells that exhibit a PD-1^{low} phenotype and result in an increased immune response.

The at least one TRL1/2 agonist and/or TLR7 agonist may be administered in combination with the immunotherapeutic agent. “Combination” refers to the ability to administer the agonist first, followed by the immunotherapeutic agent, to administer the immunotherapeutic agent first followed by the agonist, or the administration at substantially the same time of the agonist and immunotherapeutic agent.

In some embodiments, the TLR agonist is co-administered intradermal administration with the immunotherapeutic agent (e.g the TLR agonist and vaccine are co-administered). In some embodiments, the TLR agonist is formulated with the immunotherapeutic agent (e.g. vaccine) in a single formulation that is co-administered. In other embodiments, the TLR agonist and immunotherapeutic agent are formulated separately, mixed prior to administration and co-administered to the subject in a single formulation.

In some embodiments, the present disclosure provides compositions for eliciting or increasing an anti-antigen or anti-tumor response, the composition comprising at least one TLR1/2 agonist and/or at least one TLR7 agonist and at least one immunotherapeutic agent directed to the antigen or tumor (e.g., vaccine) as disclosed herein. The compositions may further comprise at least one pharmaceutically acceptable carrier. The term “pharmaceutically acceptable carrier” refers to any carrier, diluent or excipient that is compatible with the other ingredients of the formulation and not deleterious to the recipient.

As the Examples demonstrate, specific TLR agonists can increase vaccine immunogenicity and the anti-tumor response. TLR agonists treatment in combination with an immune activating agent, such as a vaccine, results in a decreased expression by the T cell of PD-1, which in turn results in an increased anti-tumor response. While TLR agonists have been previously used as stand-alone immune activating agents, potential anti-cancer therapies, and as vaccine adjuvants for the purpose of increasing innate immune stimulation, TLR agonists have not been specifically used for the purpose of reducing immune regulatory signals at the time of vaccine-directed T cell activation, as is provided here.

Prior studies of TLR agonists have shown their effects as vaccine adjuvants has been thought to be mediated by activation or recruitment of antigen presenting cells, such as B-cells or dendritic cells. Only one current publication assesses the effect of TLR stimulation on regulatory receptors, but it focuses only on TRL9. It did not assess the effects of TLR signaling on tumor treatment (Wong et al., 2009). The inventors have found that TLR1/2 or TLR7 agonists are unexpectedly more effective and have an increased anti-tumor response. TLR1/2 or TLR7 agonists have beneficial effects on anti-tumor treatment when combined with vaccination, while other TLR do not have this effect.

As further demonstrated in the Examples, TLR stimulation of APCs leads to IL-12 release that mediates reduced PD-1 expression during T-cell activation.

Aspects of the present disclosure that are described with respect to methods can be utilized in the context of the pharmaceutical compositions or kits discussed in this disclosure. Similarly, aspects of the present disclosure that are described with respect to the pharmaceutical compositions can be utilized in the context of the methods and kits, and aspects of the present disclosure that are described with respect to kits can be utilized in the context of the methods and compositions.

This disclosure provides kits. The kits can be suitable for use in the methods described herein. Suitable kits include a kit for treating a disease or condition, specifically cancers comprising a composition comprising at least one TLR1/2 agonist and/or at least one TLR7 agonist and an immunotherapeutic agent described herein. In one aspect, the kit provides composition comprising a TLR1/2 agonist and/or TLR7 agonist in amounts effective for increasing the cellular immune response to an antigen, more preferably to a tumor antigen (e.g. cancer antigen, preferably prostate cancer antigen). In some aspects, the kits provide at least one TLR1/2 and/or TLR7 agonist and at least one immunotherapeutic agent. In some aspects, instructions on how to administer the composition and/or TLR agonists and immunotherapeutic agent are provided. In some aspects, the immunotherapeutic agent is a vaccine, preferably a DNA vaccine.

Suitable kits include a kit for eliciting, increasing or improving an immune response against an antigen or immunogen. The kit comprises at least one TLR1/2 agonist and/or at least one TLR7 agonist and an immunotherapeutic agent directed against an antigen or immunogen. In some aspects, the kits provide at least one TLR1/2 agonist and/or TLR7 agonist and at least one immunotherapeutic agent. In some aspects, instructions on how to administer the composition and/or TLR agonists and immunotherapeutic agent are provided. In some aspects, the immunotherapeutic agent is a vaccine, preferably a DNA vaccine.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim.

The following non-limiting examples are included for purposes of illustration only, and are not intended to limit the scope of the range of techniques and protocols in which the compositions and methods of the present invention may find utility, as will be appreciated by one of skill in the art and can be readily implemented.

### EXAMPLES

Using the ova tumor model, the inventors have surprisingly found that stimulation of TLRs (notably using TLR1/2- or TLR7 agonists) at the time of T cell activation with an antigen, e.g. SIINFEKL (SEQ ID NO:1) peptide, can affect the magnitude of and duration of PD-1 expression on CD8+ OT-1 cells. Moreover, delivery of TLR1/2 agonist or TLR7 agonist with a DNA vaccine elicited greater anti-tumor responses to a PD-L1-expressing syngeneic tumor in vivo. Thus, regulating PD-1 through TLR at the time of T cell activation may be critical to elicit active tumor-lytic CD8+ T cells in the presence of a preexisting immunosuppressive tumor. In general, therefore, the efficacy of vaccines might be greatly augmented by targeting the upregulation or function of specific regulatory molecules at the time of T cell activation by using TLR1/2 agonist or TLR7 agonist.

### Example 1

### Activation of T Cells to Antigen in the Presence of TLR Agonist

As shown in FIG. 1A, OT-1 transgenic splenocytes were treated with high affinity antigen for Ovalbumin (OVA) (SIINFEKL, SEQ ID NO:1) in combination with Pam3CSK4 (TLR1/2 agonist) or Gardiquimod (TLR7 agonist). T cell activation was measured by the levels of marker 4-1BB (a/k/a CD137, a member of the TNF receptor superfamily). The level of PD-1 expression was also assessed in these cells (FIG. 1B). As shown, all the T cells were activated; however, the cells treated with the TLR1/2 agonist or TLR7 agonist expressed low levels of PD-1, which did not increase over time as in the cells treated with antigen alone.

### Example 2

### Tumor Growth in Mouse Tumor Model Treated with DNA Cancer Vaccine in Combination with TLR1/2 Agonist or TRL7 Agonist

Mice expressing human HLA-A2 were injected subcutaneously with SSX2-expressing sarcoma cells. Starting one day after inoculation, and every 7 days thereafter, mice were vaccinated with a DNA vaccine encoding SSX2, an optimized version of SSX2 with increased affinity for HLA-A2 (pTVG-SSX2 opt) or negative control vector pTVG4. All were done with or without TLR1/2 agonist or TLR7 agonist mixed with the DNA. A solution of DNA and a solution of TLR agonist in PBS were made and mixed together prior to administration. Note that TLR stimulation via a TLR agonist improves the efficacy of pTVG-SSX2 opt to a greater extent than pTVG-SSX2. The results of these treatments are summarized in Table 1.

TABLE 1
CR = complete response; Number = number of mice treated

| Treatment Group | CRs | Number 0 |
|---|---|---|
| 1-pTVG4 | 0 | 6 |
| 2-pTVG4 + TLR2 | 0 | 6 |
| 3-pTVG4 + TLR7 | 0 | 6 |
| 4-pTVG-SSX2 | 0 | 6 |
| 5-pTVG-SSX2 + TLR2 | 3 | 6 |
| 6-pTVG-SSX2 + TLR7 | 3 | 6 |
| 7-pTVG-SSX2 opt | 0 | 6 |
| 8-pTVG-SSX2 opt + TLR2 | 2 | 6 |
| 9-pTVG-SSX2 opt + TLR7 | 4 | 6 |

### Example 3

### Ability of TLRs to Inhibit PD-1 Expression or LAG-3 in Activated T Cells

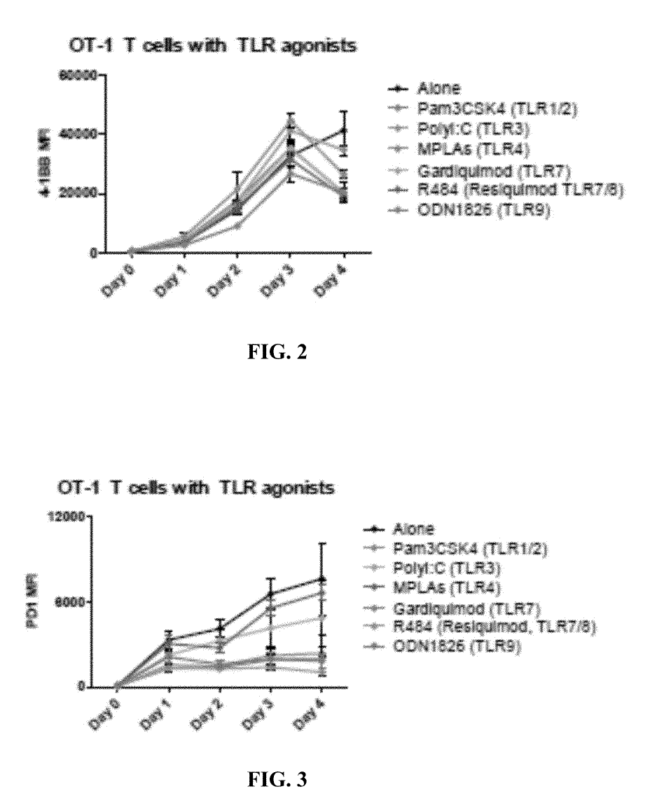

OT-1 transgenic splenocytes were treated with high affinity antigen in combination with different TLR agonists (Pam3CSK4 (TLR1 and 2 agonist), PolyI:C (TLR3), MPLAs (TLR4), Gardiquimod (TLR7 agonist), R848 (Resiquimod TLR7/8 agonist), and ODN1826 (TLR9 agonist). T cell activation was measured by the marker 4-1BB (FIG. 2). The level of PD-1 expression was also assessed in these cells (FIG. 3). Cells were also assayed for expression levels of LAG-3 (FIG. 4). As shown, all the T cells were activated when treated with the tested TLR agonist; however, the cells treated with the TLR1/2- or TLR7 agonist expressed low levels of PD-1, which did not increase over time as compared to the cells treated with antigen alone, while TLR3- and TLR4 agonists did not significantly reduce the levels of PD-1 expression in the activated T cells. There was no difference in LAG3 expression between the control cells and cells treated with any of the assayed TLR agonists.

These results are summarized in Table 2.

TABLE 2
“+” means increase, “−” means decrease, “=” means no change, “=/+” or
“=/−” means the change is there but not significant. Desired
outcomes are medium grey, neutral are light grey and bad are dark grey.
As demonstrated in Table 2, only some of the TLR agonists were able to
reduce PD-1 expression in activated T cells.

| | 4- | | | | | | |
|---|---|---|---|---|---|---|---|
| | 1BB | PD-1 | LAG3 | CD160 | CD244.2 | BTLA | VISTA |
| TLR | = | − | = | − | = | =/− | − |
| ½ | | | | | | | |
| TLR 3 | = | =/− | =/+ | | | | |
| TLR 4 | = | = | = | | | | |
| TLR 7 | = | − | = | − | =/− | =/− | = |
| TLR | = | − | = | | | | |
| ⅞ | | | | | | | |
| TLR 9 | = | − | =/− | | | | |

### Example 4

### Effect of TLR1/2- and TLR7 Agonists on Different Checkpoint Markers

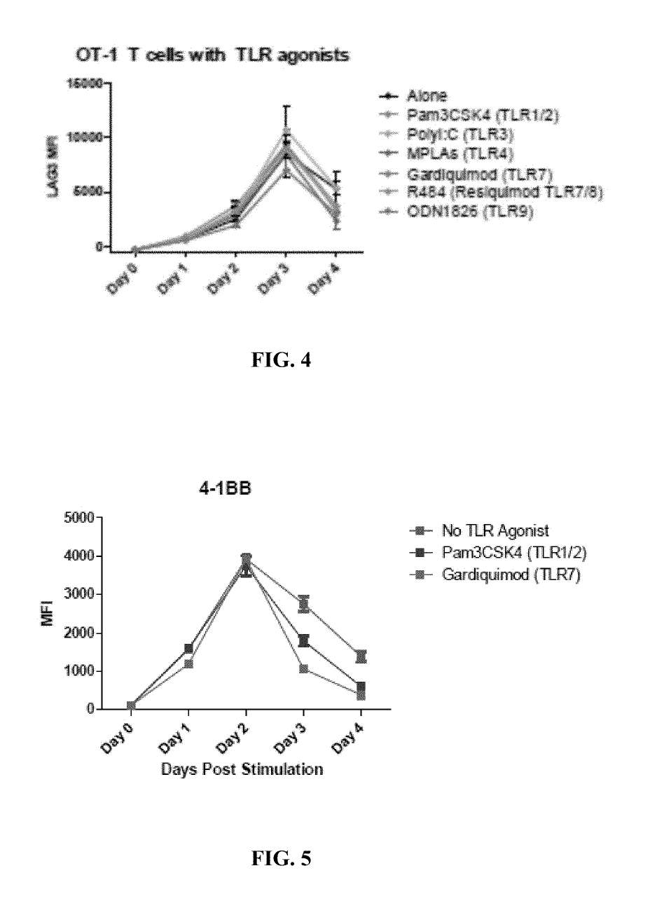

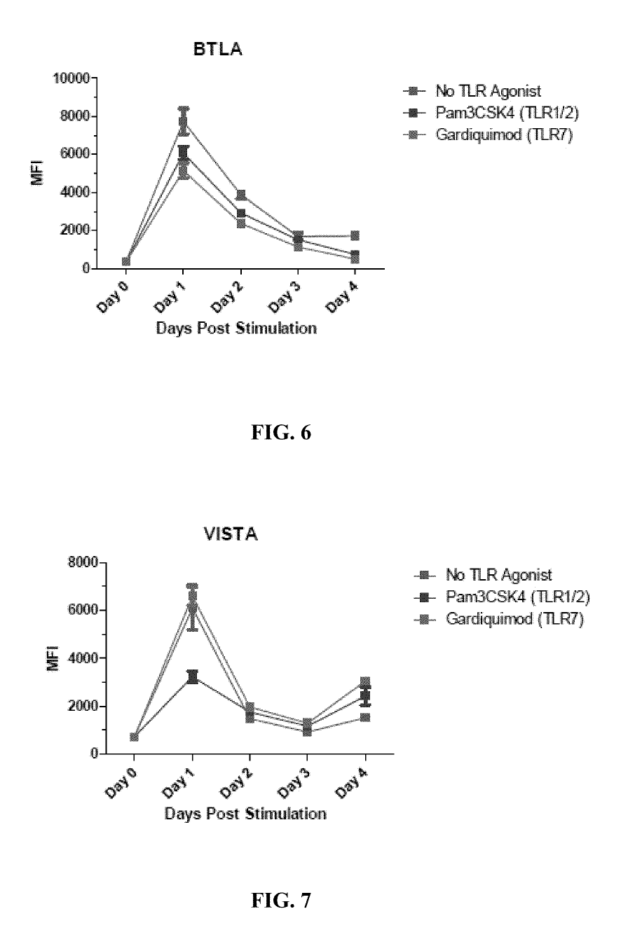

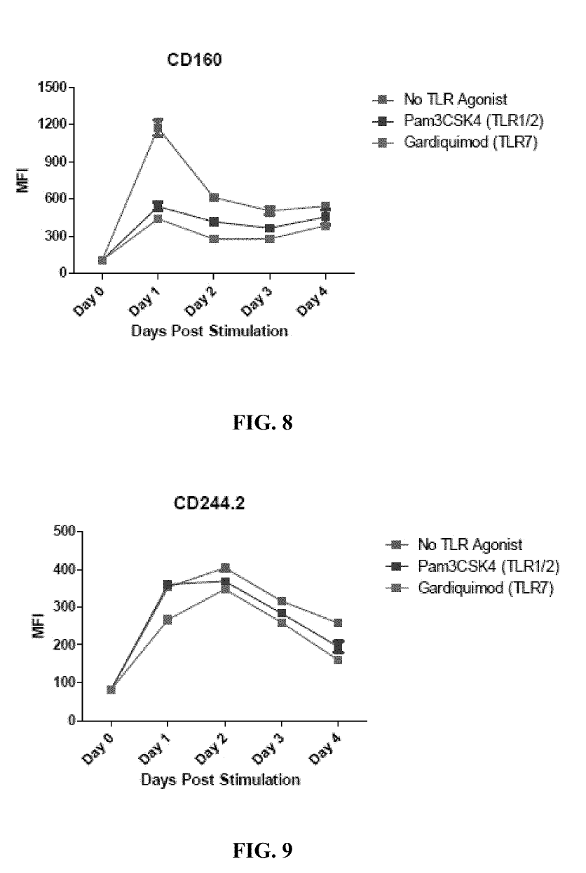

OT-1 transgenic splenocytes were treated with high affinity antigen in combination with Pam3CSK4 (TLR1 and 2 agonist), Gardiquimod (TLR7 agonist) or no TLR agonist and the levels of different checkpoint markers or T cell stimulation (measured by 4-1BB, FIG. 5) were assayed. The checkpoint markers BTLA (FIG. 6), VISTA (FIG. 7), CD160 (FIG. 8) and CD244.2 (FIG. 9) were assayed. As demonstrated, TLR1/2- and TLR7 agonists did not affect the levels of any of the tested checkpoint markers except PD-1 (FIG. 1B).

### Summary:

A report by Olino et al. demonstrated that treatment of mice with established hepatic metastases using a tumor antigen-expressing recombinant Listeria monocytogenes (Lm) vaccine elicited cytotoxic CD8+ T cells that had decreased expression of PD-1, but with variable effects on CTLA-4 expression [32]. Similarly, Drake and colleagues have demonstrated in a tolerant murine system (expressing HA) that immunization with a Lm vaccine encoding HA did not induce PD-1 expression relative to other methods of immunization [33]. These findings, in addition to those described above, suggest that the PD-1 expression can be independent of T cell activation, or can be at least modulated at the time of T cell activation.

The impact of TLR activation on CD8+ T cells stimulated with high-affinity versus low-affinity epitopes has not been rigorously evaluated with respect to expression of these regulatory molecules. In studies using the same Ova/OT-1 in vitro model, the inventors investigated the effects of TLR activation on PD-1 expression following T cell activation with the high-affinity SIINFEKL (SEQ ID NO:1) epitope. As shown in FIG. 2, T cell activation in the presence of TLR2- or TLR7 agonists resulted in T cell activation but with low PD-1 expression. Importantly, when TLR2- or TLR7 agonists were delivered with a high MHC-affinity peptide vaccine in vivo, they improved the anti-tumor response similar to PD-1 blockade (FIG. 1C).

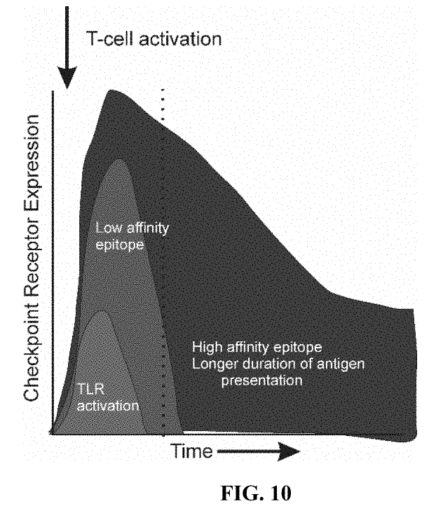

Taken together, our results demonstrate that increasing activation of T cells leads to expression on activated CD8+ T cells of regulatory molecules, notably PD-1. This increased expression of PD-1 results in a higher likelihood of the T cell being inactivated and a reduced anti-tumor response. This activation-induced upregulation/transient expression of PD-1 has been relatively overlooked by the tumor immunology field. In fact, there have been many efforts to use epitopes with high MHC affinity either as direct immunogens, or encoded within vectors. Our results demonstrate that the prior approach of using high affinity epitopes will be less effective in the absence of efforts to either block PD-1 signaling or potentially to reduce expression of PD-1 at the time of T cell activation. Further, our findings in animal models and an ongoing clinical trial demonstrate that blocking PD-1 ligation shortly after T cell activation is sufficient to improve the anti-tumor response, suggesting that efforts to block T cell regulation (and deactivation) at the time of immunization are critical. A model (FIG. 10) is proposed that serves as the basis for the studies.

### Example 5

### TLR1/2 and/or TLR7 Stimulation at the Time of T Cell Activation via Anti-Tumor Immunization Elicits Higher Frequencies of Antigen-Specific Effector and Memory CD8+ T Cells and Greater Anti-Tumor Efficacy in Murine Models

As shown in Example 1 above (FIG. 1C), co-treatment with certain TLR agonists at the time of OT-1 T cell stimulation with SIINFEKL (SEQ ID NO:1) peptide elicited activated T cells with lower PD-1 expression. Delivering a TLR antagonist in vivo in combination with a DNA vaccine that encodes a high-affinity epitope resulted in anti-tumor response greater than compared with DNA vaccine alone, and as great as combining immunization with PD-1 blockade (FIG. 1C). These findings suggest that an alternative to PD-1 blockade, or other checkpoint molecule blockade, at the time of T cell activation with an immunotherapeutic agent (e.g. vaccine) is to use agents that might lower expression of these molecules (see model, FIG. 10). While TLR activation has been known for many years to help activate CD8+ T cells, mechanisms underlying this have not been well understood

### Example 6

### TLR Stimulation Lowers PD-1 on CD8+ T-Cells Mediated by IL-12

This Example demonstrates that specific TLR agonists (specifically agonists for TLR1/2, TLR7, and TLR9) are able to increase the anti-antigen response, specifically the antitumor response of activated immune cells (CD8+ T cells). This Example shows that treatment using the TLR agonists results in decreased expression of PD-1 on antigen-activated naïve T-cells which in turn leads to the increased anti-antigen response and a reduced and inhibited tumor growth in the in vivo cancer cell model. These effects were mediated by IL-12 released by professional antigen-presenting cells. Gene expression analysis demonstrated that T cells activated in the presence of either TLR1/2 or TLR7 agonists had similar transcriptional profiles associated with increased effector function.

In two separate tumor models, treatment with anti-tumor vaccines in the presence of TLR1/2 or TLR7 ligands induced antigen-specific CD8+ T cells with lower PD-1 expression and improved anti-tumor immunity.

This Example sought to determine if TLR stimulation, at the time of T-cell activation, leads to changes in T-cell checkpoint molecule expression and whether this might be used to improve the efficacy of anti-tumor vaccines. A well-characterized OT-1 model system was used in this Example, activating OT-1 CD8+ T cells with high- or low-affinity peptide ligands. We found that agonists for TRL1/2, 7, 7/8, and 9, but not TRL3 or 4, can reduce activation-induced PD-1 expression on CD8+ T cells. This effect was mediated by both B cells and dendritic cells (DCs) as antigen-presenting cells (APC), did not rely on CD80 or CD86 co-stimulatory signals, and was dependent upon IL-12 release by APC. Gene expression analysis demonstrated that the resulting T cells harbored fewer PD-1 transcripts, and had increased expression of genes associated with cytotoxic activity, Th1cytokines, and IL-12 receptors. Furthermore, in two different tumor models using either a peptide or DNA anti-tumor vaccine, the use of specific TLR ligands as adjuvants led to reduced PD-1 expression on vaccine-activated CD8+ T cells and increased anti-tumor responses. These findings demonstrate the importance of TLR stimulation at the time of T-cell activation to elicit an effective adaptive anti-tumor T-cell response. Moreover, these data support the use of defined TLR agonists as vaccine adjuvants by exploiting their ability to modulate expression of specific T-cell checkpoint molecules at the time of T-cell activation.

Results

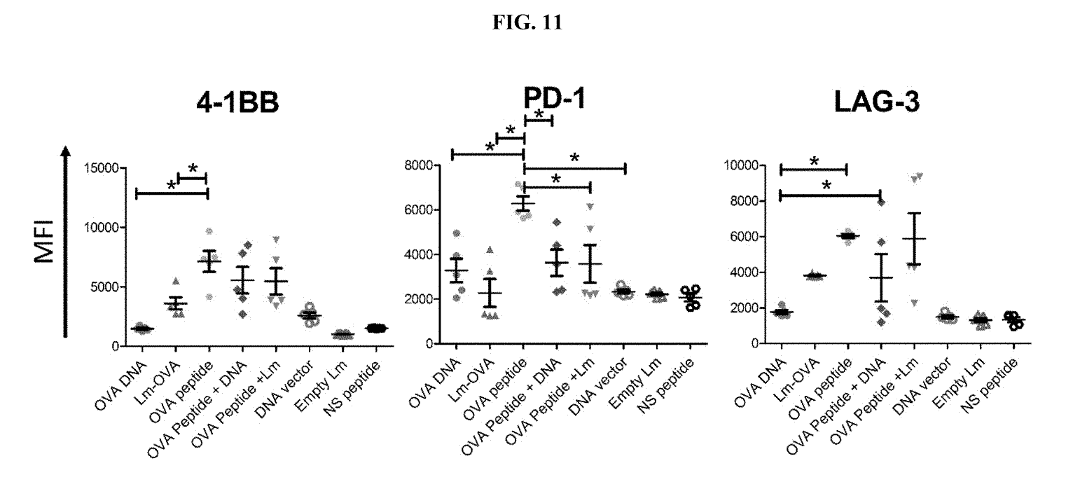

Vaccine type can affect expression of T-cell checkpoint molecules on activated CD8+ T cells: This Example used an ovalbumin vaccination model. OT-1 cells were adoptively transferred to naïve C57BL/6 mice. The day after transfer, mice were immunized once with (a) DNA encoding ovalbumin, (b) Listeria monocytogenes (Lm) encoding ovalbumin, (c) OVA₂₅₇₋₂₆₄ peptide (SIINFEKL (SEQ ID NO:1)), (d) SIINFEKL peptide (SEQ ID NO:1) with DNA vector, (e) DNA vector alone, (f) Lm encoding control vector with no antigen, or (g) NS (non-specific) peptide. Splenocytes were collected the day after vaccination for expression of cell surface markers on antigen-specific OT-1 cells. As shown in FIG. 11, the strongest activation, as measured by 4-1BB expression, occurred with peptide immunization. Expression of PD-1 and LAG-3 were, as expected, significantly increased with peptide immunization. 4-1BB expression was similarly increased in the presence of Lm or DNA co-administered at the site of peptide immunization. PD-1 expression, however, was significantly reduced in the presence of Lm or DNA. LAG-3 expression was not reduced in the presence of Lm co-administered with peptide. Together, these findings suggested that different vaccine types, potentially due to innate immune activation properties of DNA or Lm vectors, might affect expression of T-cell checkpoint molecules independent of T-cell activation.

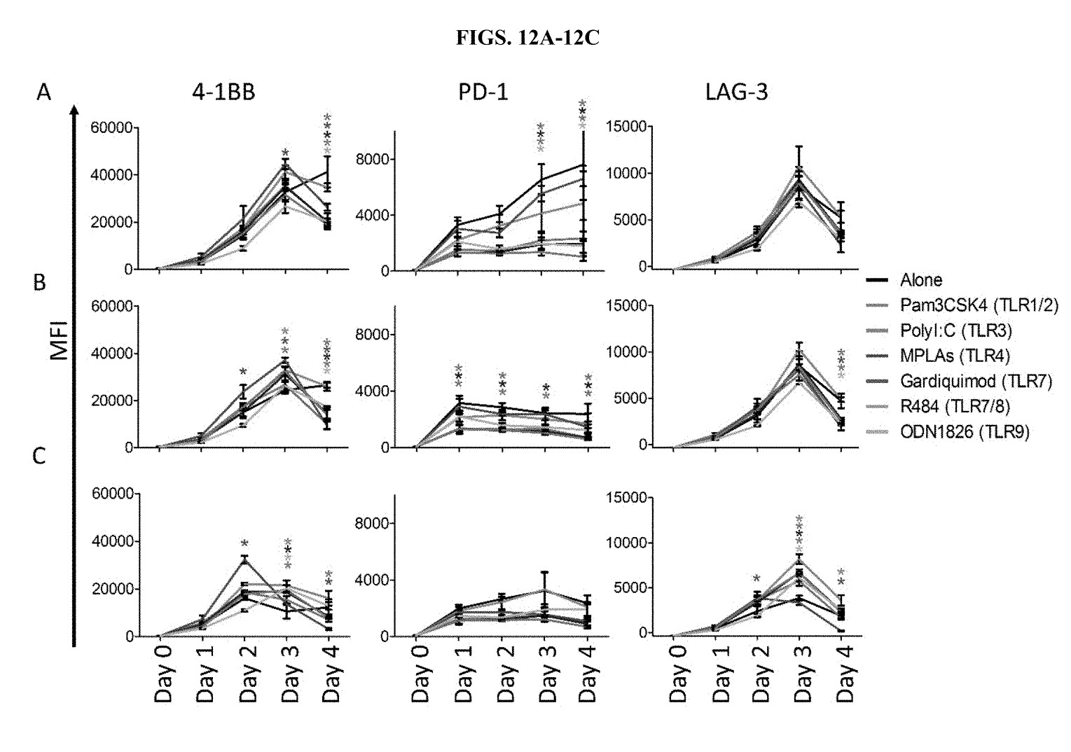

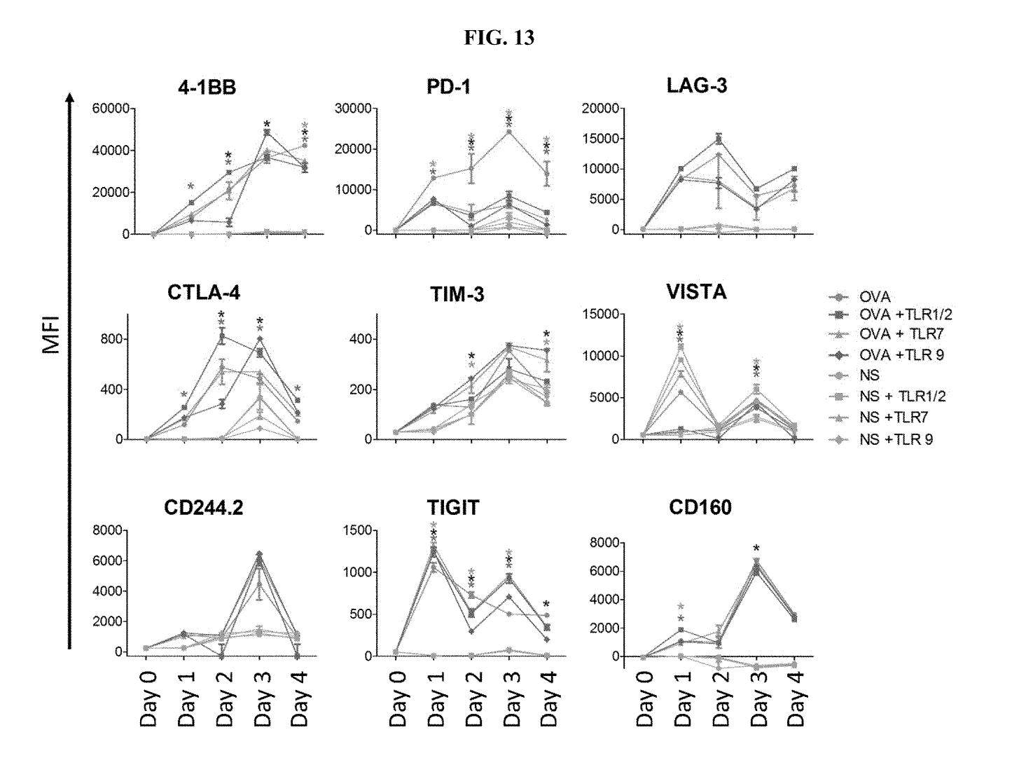

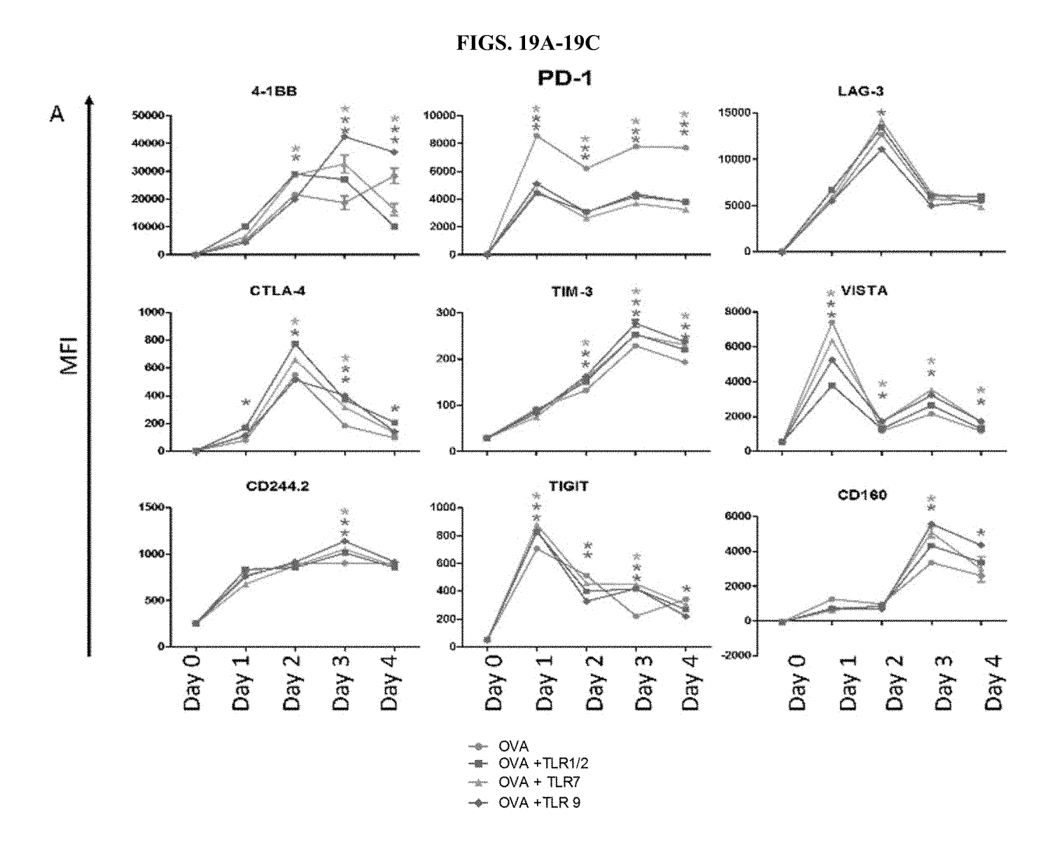

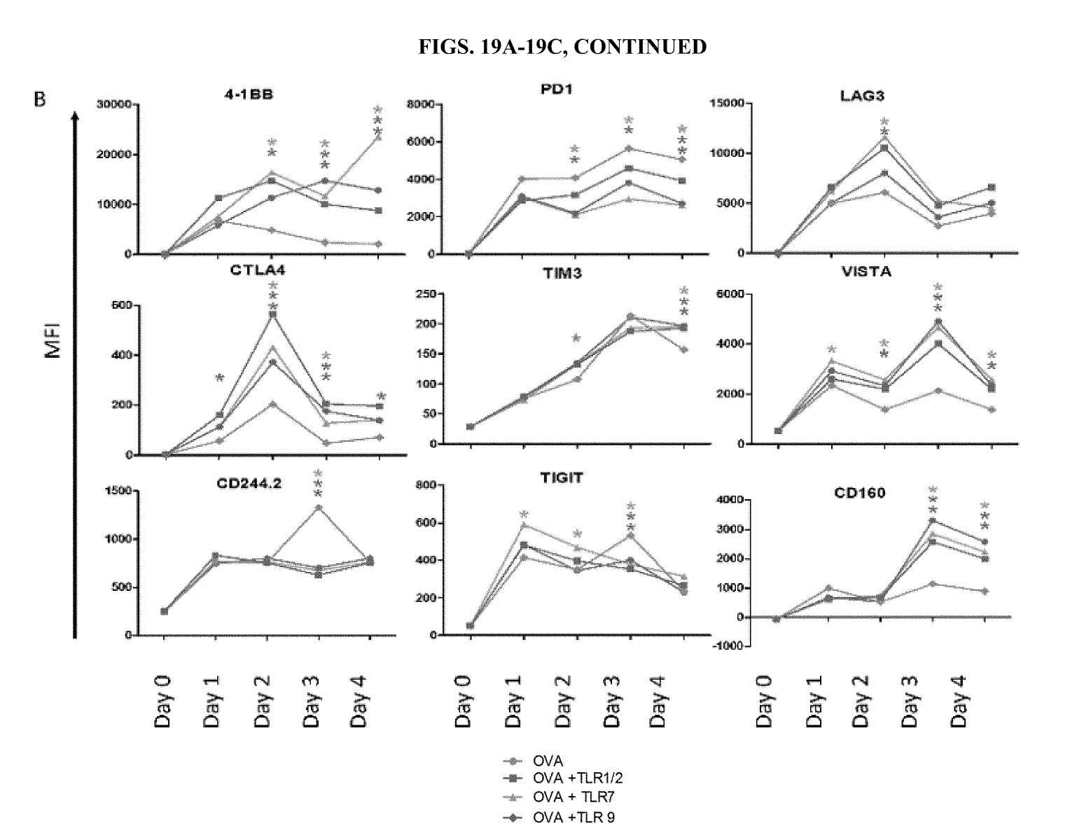

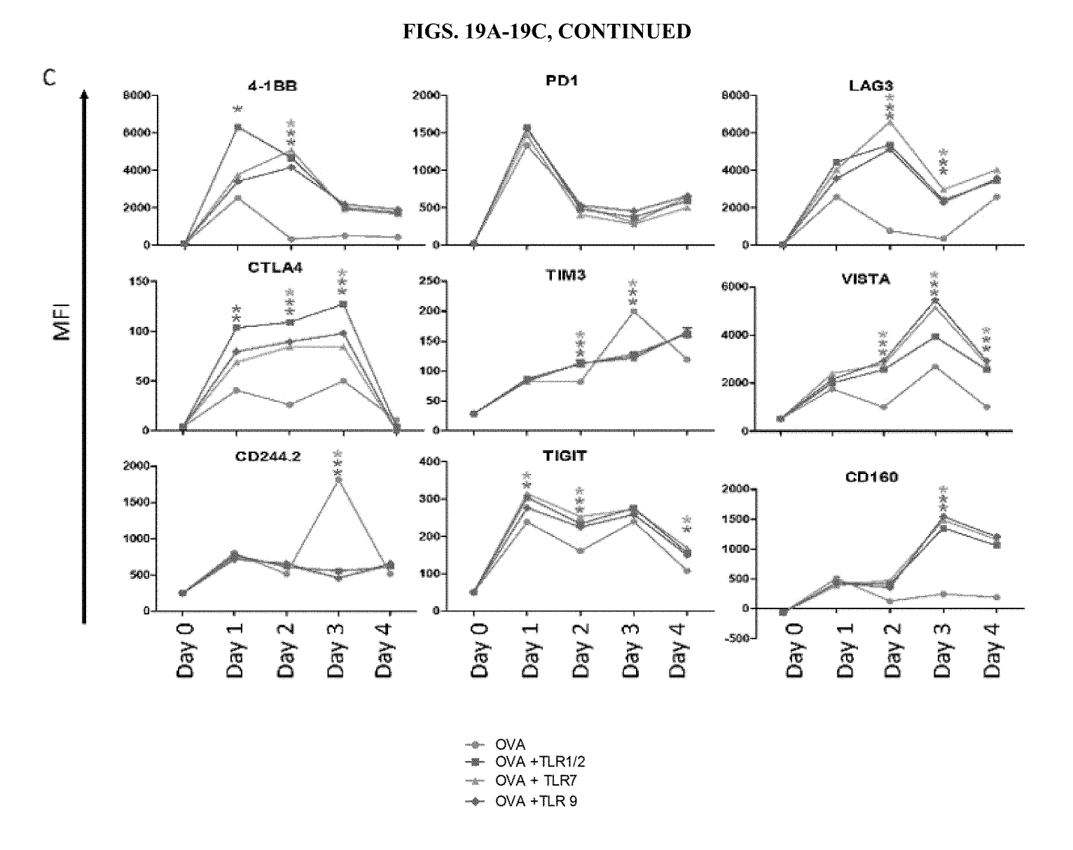

TLR stimulation at the time of T-cell activation in vitro affects expression of PD-1. Given that CpG-rich DNA is a TLR9 agonist and Lm is a TLR2 agonist, we questioned whether TLR stimulation with different ligands could similarly affect expression of different T-cell checkpoint inhibitors. To test this, we employed the same OT-1 model using peptide antigens with different MHC binding affinities, given our previous findings that high affinity antigens caused greater activation of CD8+ T cells, but also elicited significantly higher expression of PD-1 relative to lower affinity antigens (8). OT-1 splenocytes were stimulated in vitro with peptides of different affinity for MHC class I (high-affinity SIINFEKL peptide (SEQ ID NO:1, OVA), moderate-affinity SIINTEKL peptide (SEQ ID NO:2, FT), or low-affinity SIINFEKP peptide (SEQ ID NO:7, LP)), alone or in the presence of different TLR ligands (TRL1/2 (Pam3CSK4), TRL3 (Poly I:C), TRL4 (MPLAs), TRL7 (Gardiquimod), TRL7/8 (R848), or TRL9 (ODN1826)). As demonstrated in FIG. 12, OVA peptide elicited the greatest T-cell activation as measured by 4-1BB expression, and this was not affected over 4 days by the presence of different TLR ligands. Similarly, LAG-3 expression increased by day 3, but was not affected by the presence of different TLR ligands. PD-1 expression, however, was significantly reduced in the presence of TLR1/2, TLR7, TLR7/8, and TLR9 ligands. This reduction in PD-1 expression was observed most with the high-affinity SIINFEKL (SEQ ID NO:1) epitope, but also observed following activation with lower affinity epitopes. Given these findings, we evaluated expression of multiple other T-cell checkpoint molecules following T-cell activation in the presence of TLR ligands. As shown in FIG. 13, OT-1 T cells activated with SIINFEKL (SEQ ID NO:1) in the presence of TLR1/2, TLR7, and TLR9 ligands predominantly affected expression of PD-1 relative to LAG-3, TIM-3, VISTA, CD244, TIGIT, or CD160. Cytotoxic T-lymphocyte-associated protein 4 (CTLA-4) expression, however, was increased in the presence of TLR1/2 agonist. Similar results were found activating OT-1 cells with the lower affinity FT (SIINTEKL peptide (SEQ ID NO:2)), LP (SIINFEKP peptide (SEQ ID NO:7), or LY (SIINFEKY, SEQ ID NO:8)epitopes (FIG. 19).

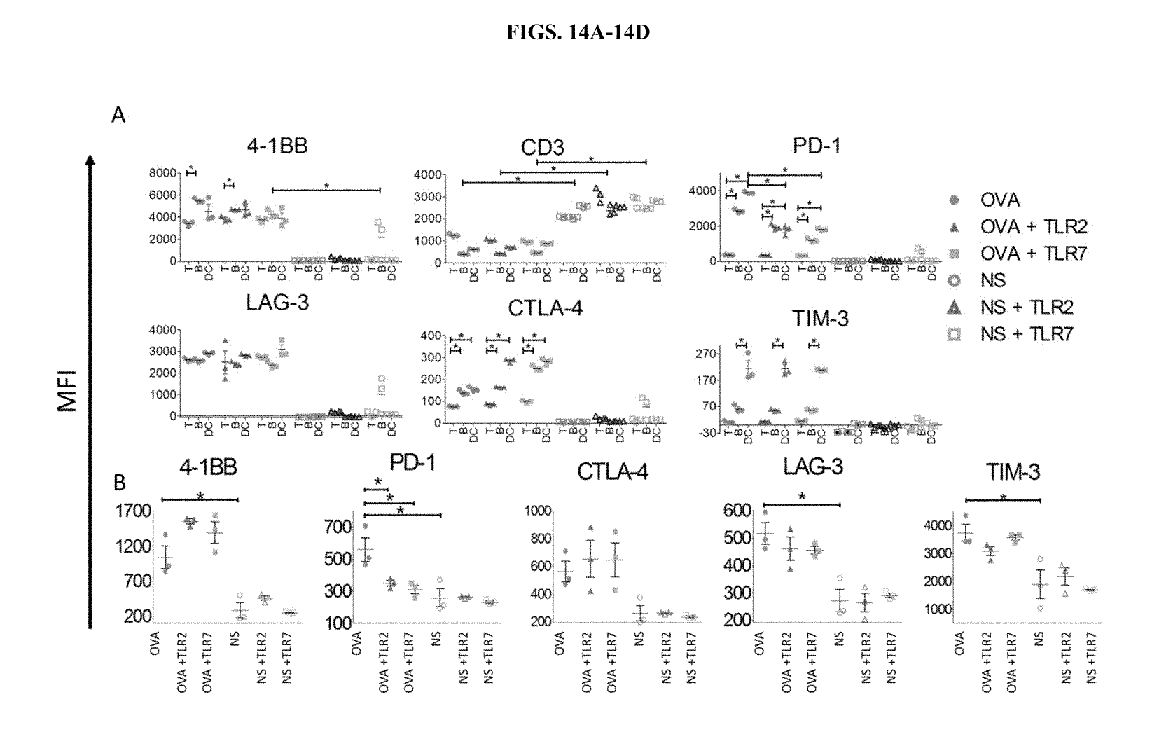

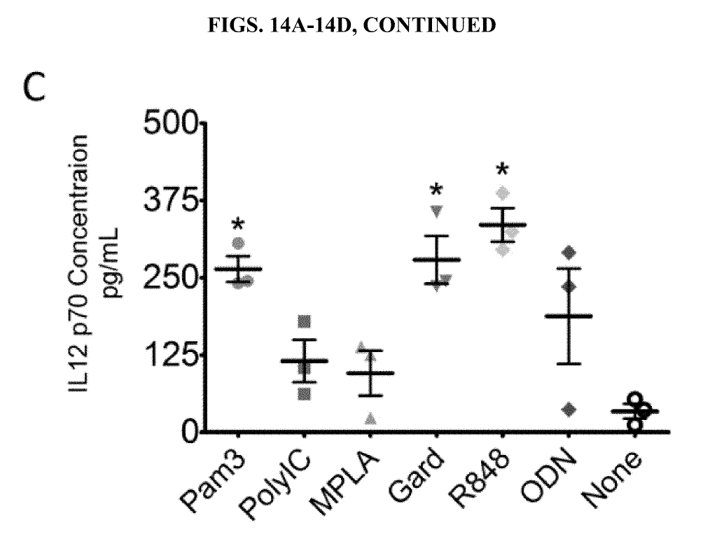

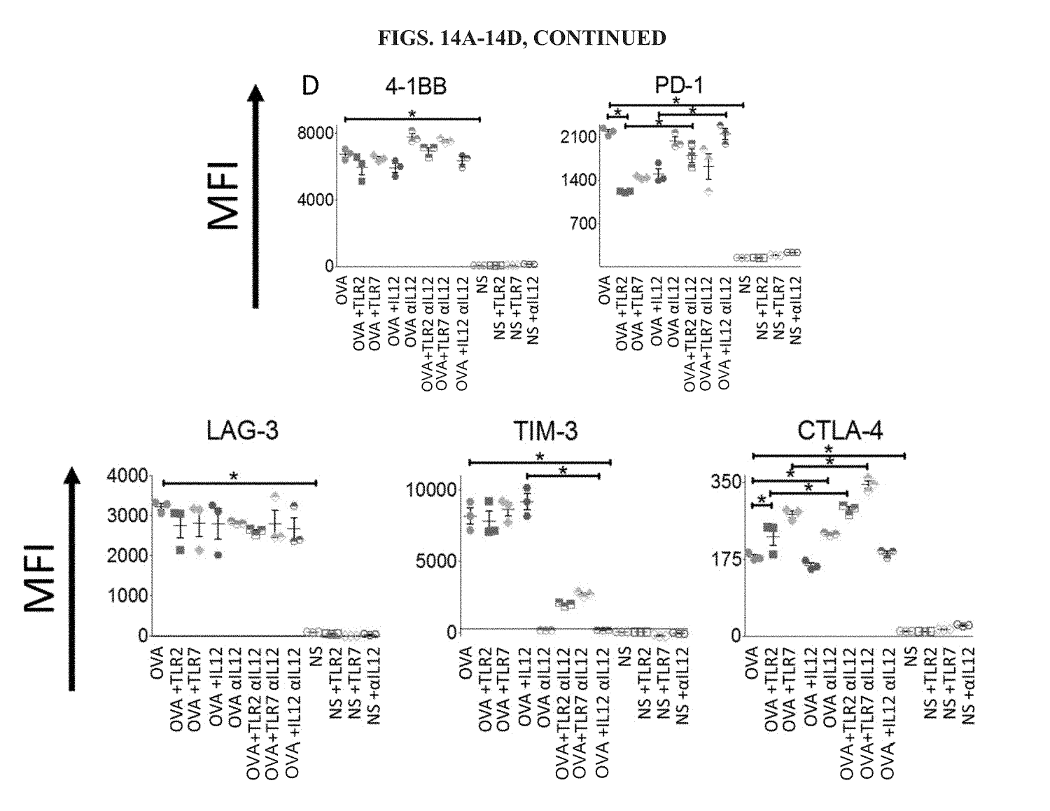

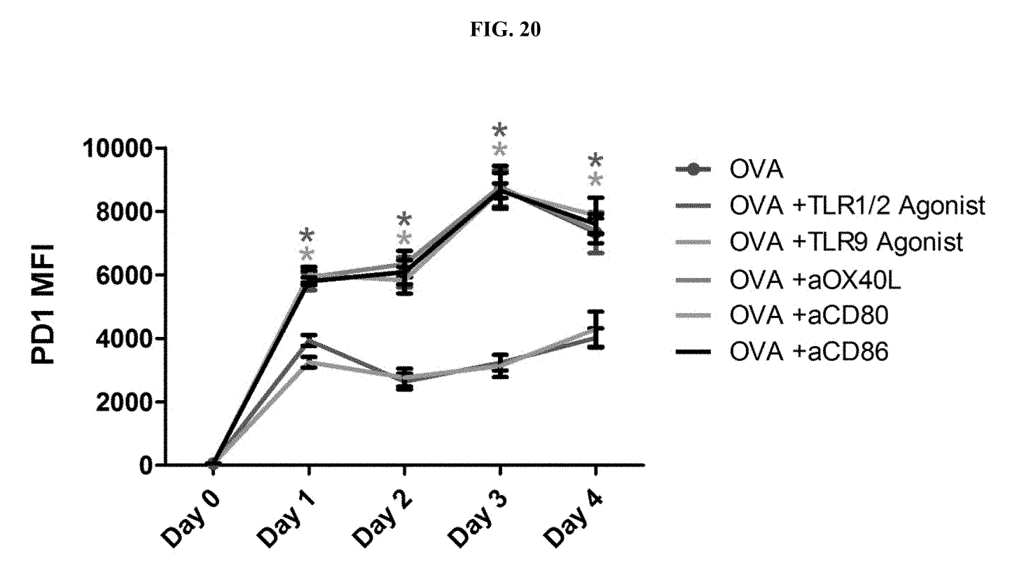

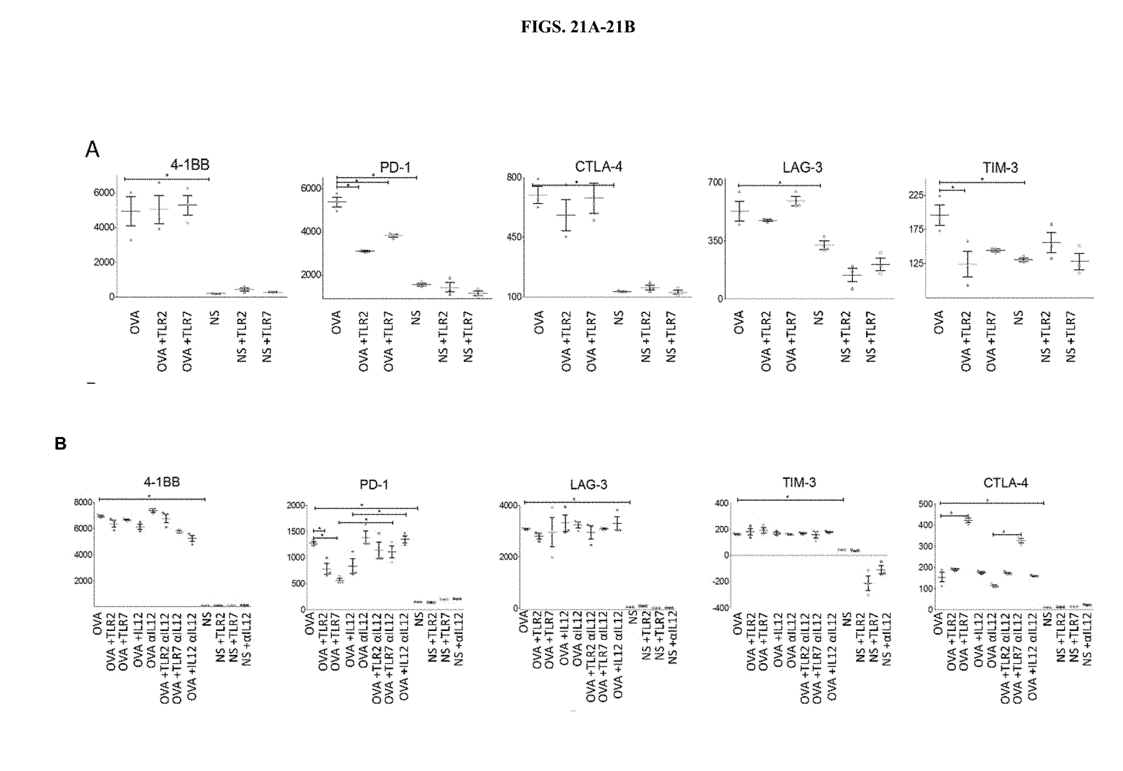

Changes in PD-1 expression following TLR stimulation are mediated by IL-12 secreted from APCs: TLR are expressed by both T cells and professional APC. To determine if the effects on T-cell checkpoint expression were mediated by direct effects of TLR ligands on T cells or APC, purified OT-1 T cells were activated with SIINFEKL peptide (SEQ ID NO:1) alone or in the presence of TLR1/2 or TLR7 agonist, or with purified DC or B cells pre-treated with TLR agonists. After 24 hours cells were evaluated for expression of 4-1BB, CD3, PD-1, LAG-3, CTLA-4, and TIM-3. As shown in FIG. 14, cells were activated in the presence or absence of professional APC, as measured by 4-1BB expression. Expression of LAG-3 was not affected by the APC type or the presence of TLR ligand, and TIM-3 expression, while dependent on DC as the APC type, was not affected by the presence of TLR ligand. PD-1 expression, however, required professional APC (either B cells or DC), and was reduced in the presence of either TLR ligand. Blockade of CD80 or CD86, however, did not affect PD-1 expression on OT-1 T cells activated with the SIINFEKL epitope (SEQ ID NO:1), suggesting that APC signals other than these co-stimulatory molecules were required for PD-1 expression (FIG. 20). To determine if these APC signals were due to cell-to-cell receptor-ligand mediated interaction, or due to a secreted factor, conditioned medium from APC incubated with or without TLR ligands was used in place of the TLR agonist during T-cell stimulation. As shown in FIG. 14B, OT-1 T cells activated by professional APC in the presence of TLR conditioned media showed a reduction in PD-1 like that observed with TLR agonist treatment directly, suggesting the decrease of PD-1 expression was due to release of a secreted factor by TLR-stimulated APC. A recent report by Yin and colleagues demonstrated that CpG treatment of mice led to CD8+ T cells with lower expression of PD-1, an effect dependent on IL-12 signaling (17). Consequently, we tested whether treatment of APC with TLR ligands elicited production of IL-12. As shown in FIG. 14C, treatment of DC with TLR1/2 and TLR7 agonists led to the greatest secretion of IL-12. No significant secretion of IL-12 was detected following treatment with TLR3 or TLR4 agonists. Moreover, as shown in FIG. 14D, OT-1 T cells activated with peptide in the presence of IL-12 had lower PD-1 expression. Similar results were found if B cells were used in place of DC as APC (FIG. 21). Finally, the reduction of PD-1 expression on OT-1 T cells activated in the presence of TLR-stimulated APC was abrogated in the presence of IL-12 blockade. These findings demonstrate that TLR stimulation of APC leads to IL-12 release that mediates reduced PD-1 expression during T-cell activation.

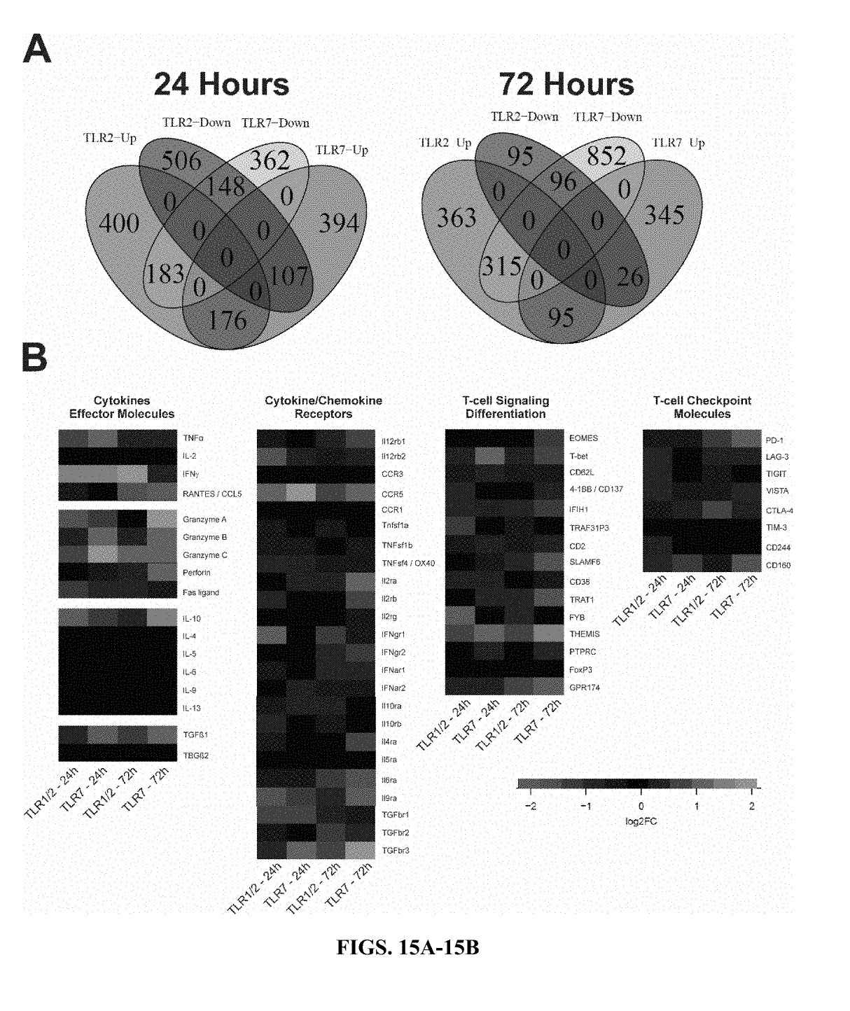

T-cells activated in the presence of TLR1/2 or TLR7 ligands exhibit a gene expression profile consistent with enhanced effector function: To characterize gene expression changes in CD8+ T cells activated in the presence of TLR ligands, and to determine whether the effects of TLR1/2 and TLR7 activation were entirely redundant, OT-1 splenocytes were activated for 24 to 72 hours in the presence of SIINFEKL peptide alone (SEQ ID NO:1) or with TLR1/2 (Pam) or TLR7 (Gardiquimod) ligands. CD8+ OT-1 T cells were then purified and evaluated for gene expression changes by RNA sequencing (RNA-seq). As shown in FIG. 15, OT-1 T cells activated in the presence of either TLR1/2 (Pam3CSK4) or TLR7 (Gardiquimod) ligands showed similar but distinct enriched GO-term expression changes when compared to cells activated by peptide antigen alone. Both TLR ligands induced expression of genes associated with Th1-biased cytotoxic effector function (including IFNγ, TNFα, granzymes A-C, perforin and T-Bet), reduced Eomesodermin (EOMES gene) expression, and reduced expression of genes associated with T-cell regulation (including PD-1 and IL-10) when compared to cells stimulated with peptide alone.

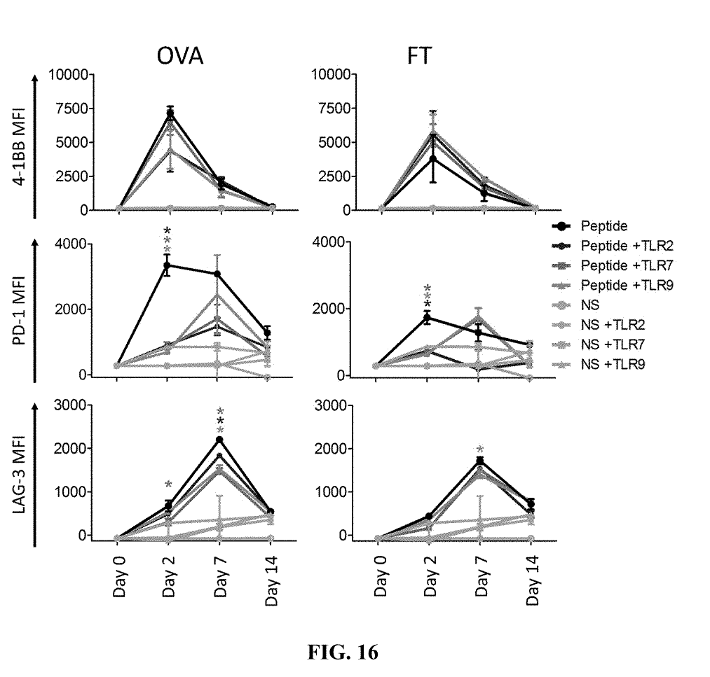

TLR stimulation at the time of T-cell activation reduced expression of PD-1 in vivo: To determine whether TLR stimulation at the time of T-cell activation with vaccination elicited a similar reduction in PD-1 in vivo, OT-1 cells were adoptively transferred to naïve C57BL/6 mice as above, and immunized once with OVA or FT peptide (or non-specific peptide) one day later in the presence or absence of a TRL1/2, TLR7, or TLR9 ligand. Splenocytes were collected from groups up to 14 days after immunization and evaluated for expression of 4-1BB, PD-1 and LAG-3. As demonstrated in FIG. 16, 4-1BB expression and LAG-3 increased after immunization and their expression was not affected by concurrent delivery of one of the TLR agonists. PD-1 expression, however, was significantly reduced early after T-cell activation.

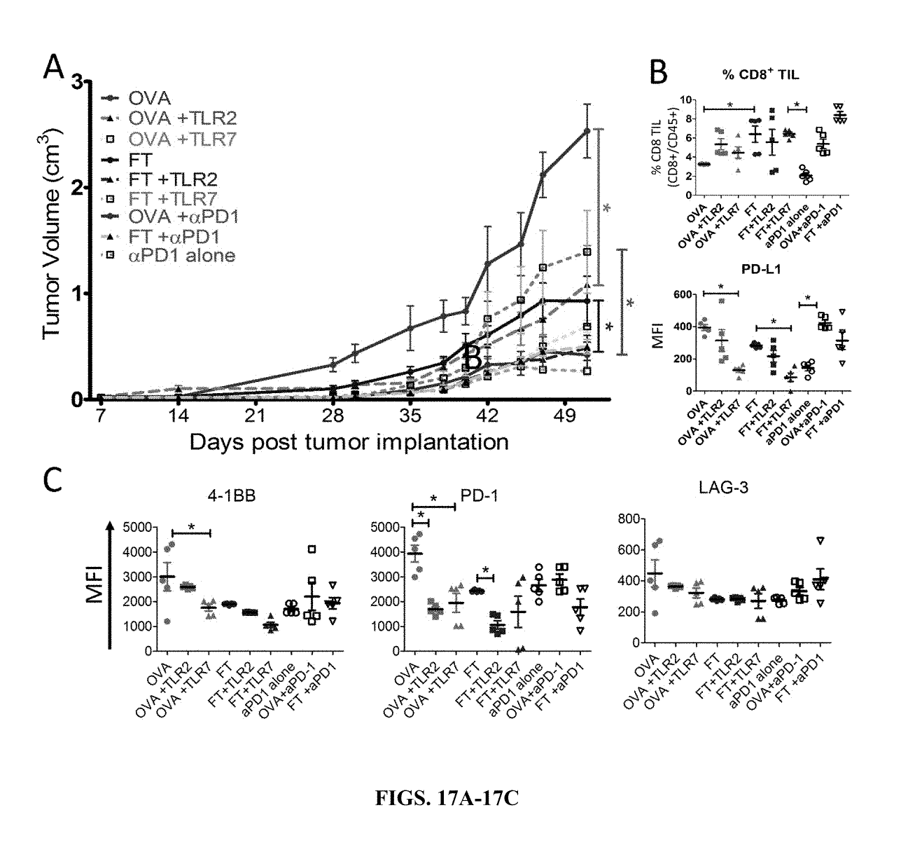

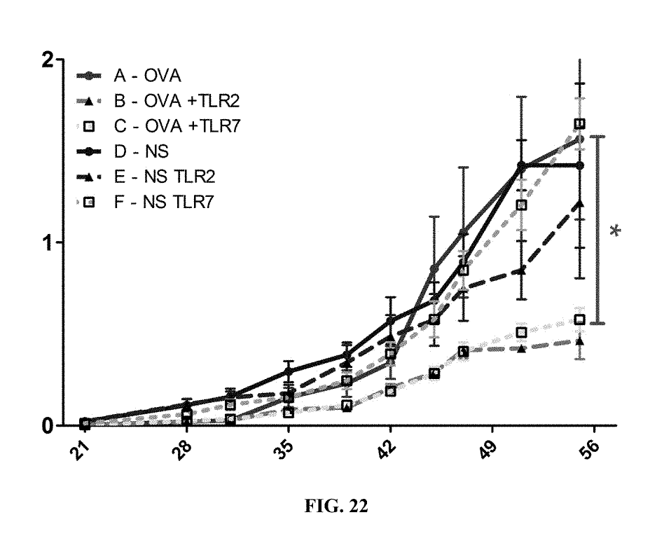

Immunization in the presence of TLR1/2 or TLR7 ligands, co-delivered as vaccine adjuvants, elicits greater anti-tumor immunity in vivo: To determine whether the reduced expression of PD-1 on T-cells activated in the presence of TLR stimulation could mediate greater anti-tumor response, ovalbumin-expressing E.G7 cells were implanted in C57BL/6 mice and permitted to grow until palpable (14 days). OT-1 T cells were then adoptively transferred and the following day were immunized with SIINFEKL (SEQ ID NO:1, OVA) or FT peptide (SEQ ID NO:2) alone or with TLR1/2 or TLR7 agonist. Other groups were immunized with 100 μg anti-PD-1 antibody delivered the following day. In this model, PD-1 blockade alone was only moderately effective in the absence of activation of T cells with vaccines, as we have previously reported (8). As shown in FIG. 17A, while immunization with FT peptide elicited a greater anti-tumor response relative to the high-affinity OVA peptide, immunization with either peptide in the presence of TLR1/2 or TLR7 agonist produced a greater anti-tumor effect. Notably, the combination of vaccine with TLR agonists was at least as effective as combination with PD-1 blockade. This anti-tumor effect was not observed with TLR agonist treatment alone (FIG. 22). As shown in FIG. 17B, immunization with TLR1/2 (Pam3CSK4) or TRL7 (Gardiquimod) agonists elicited a greater number of CD8+ tumor-infiltrating lymphocytes, and reduced PD-L1 expression on tumor cells. This was associated with a reduced PD-1 (but not LAG-3) expression on CD8+ TIL (FIG. 17C).

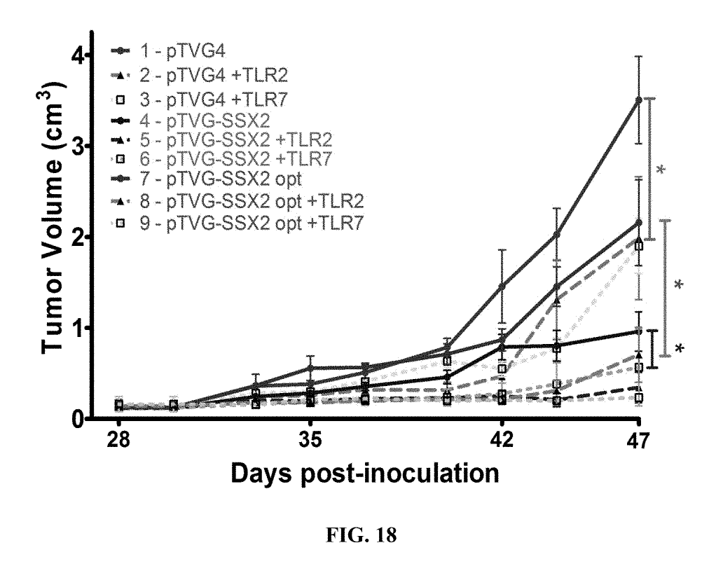

Similar findings were observed in a separate tumor model using a DNA vaccine. We have previously reported that a DNA vaccine encoding an antigen expressing epitopes with high affinity for MHC class I elicited antigen-specific CD8+ T cells expressing higher PD-1 and an inferior anti-tumor response (9). Using this model, HLA-A2-expressing transgenic mice were implanted with synovial sarcoma, X breakpoint 2 (SSX-2)-expressing tumor cells. After 2 days, mice were then immunized weekly with control vector (pTVG4), DNA encoding native SSX2 (pTVG-SSX2), or DNA encoding high affinity HLA-A2 epitopes (pTVG-SSX2 opt) in the presence or absence of Pam3CSK4 (TLR1/2) or Gardiquimod (TLR7) agonist. As shown in FIG. 18, while TLR1/2 or TLR7 agonists alone (with vector DNA) had modest effects on tumor growth, in combination with either DNA vaccine they significantly increased the anti-tumor efficacy.

In this Example, we investigated whether expression of PD-1, and other T-cell immune checkpoint molecules, at the time of T-cell activation can be reduced in the context of innate immune stimulation using TLR ligands. Using in vitro models, we found that specific TLR ligands, notably ligands for TLR1/2, TLR7 and TLR9, led to lower expression of PD-1 on activated CD8+ T cells, with less effect on expression of other T-cell checkpoint molecules. This was due to effects on professional antigen presenting cells, either dendritic cells or B cells, and was not mediated through expression of CD80 or CD86 on APC, or CD40 on T cells. Rather, this was mediated by IL-12 release from APC following TLR stimulation. In vivo use of these TLR ligands at the time of T-cell activation with vaccines led to superior anti-tumor immunity in tumor systems regulated by the PD-1/PD-L1 pathway. Our findings provide mechanistic understanding of ability of TLRs to be used as vaccine adjuvants due to their effects on IL-12 release by APC and PD-1 expression on CD8+ T cells, and that specific TLR ligands serve as optimal anti-tumor vaccine adjuvants. Our results also provide some of the first evidence that PD-1 expression can be separated from T-cell activation, a finding that has important implications for T-cell therapies for cancer. Finally, our gene expression results suggest that TLR stimulation may modulate the development of effector and memory function in CD8+ T cells leading to more effective vaccine approaches.

Our findings demonstrate that the adjuvant effects of specific TLRs are specific for PD-1 compared with other immunomodulatory molecules expressed with T-cell activation, and are mediated by IL-12.

In the context of anti-tumor immunity this is of great importance, as we have previously demonstrated that even short-term increase in PD-1 expression following T-cell activation by vaccination can impair anti-tumor immunity (34). We demonstrate here that using specific TLR stimulation to decrease PD-1 expression following T-cell activation with vaccines can lead to anti-tumor responses at least as effective as are obtained using vaccines combined with PD-1 blockade directly.

Our gene expression analysis suggests that TLR1/2 and TLR7 stimulation activate similar T-cell transcriptional profiles, hence their effects on PD-1 expression may be redundant. Our studies demonstrate that this reduction of PD-1 expression was mediated by IL-12. While other groups have demonstrated that certain TLR agonists (notably TRL7, but not TLR3 or TLR4) can lead to IL-12 production by DC, our report is the first, to our knowledge, specifically linking this to expression of PD-1 on activated T cells (35-37).

Despite the similar effects of either TLR1/2 or TLR7 stimulation on PD-1 expression, curiously we found these led to different effects on CTLA-4 expression (FIGS. 13 and 14). This suggests that they do not activate completely redundant pathways. Moreover, while TLR3 activation did not lead to decreased PD-1 expression, and TLR3 is the only TLR not using the downstream MyD88 adapter protein, the finding that TLR4 activation also did not lead to PD-1 downregulation suggests that this effect could be independent of MyD88 signaling.

Our findings demonstrate that specific TLR stimulation can affect the transcriptional profile of CD8+ effector cells, potentially favoring establishment of effector memory cells with high expression of T-Bet and lower expression of EOMES. Our findings further suggest that different TLR agonists might be strategically used, alone or in sequence with other cytokines, to increase the anti-tumor efficacy of CD8+ effector memory cells with vaccination.

Materials and Methods:

Mice: HLA-A2.01/HLA-DR1-expressing (HHDII-DR1) mice on a C57BL/6 background were obtained from Charles River Labs courtesy of Dr. Francois Lemonnier (41). OT-1 (Stock No: 003831) and C57BL/6J (B6, Stock No: 000664) were purchased from The Jackson Laboratory (Jax, Bar Harbor, Mass.). All mice were maintained and treated in microisolator cages under aseptic conditions and all experiments were conducted under an IACUC-approved protocol that conforms to the NIH guide for the care and use of laboratory animals.

Cell Lines: E.G7-OVA (CRL-2113) cells were obtained from ATCC (Manassas, Va.) and maintained via the ATCC recommended methods. E.G7-OVA cells were lentivirally transduced to express PD-L1, as previously described (34). The A2/sarcoma cell line expressing SSX2 (A2/Sarc-SSX2) was generated as previously described (42).

Peptides: Peptides encoding the high-affinity H2K^{b}-restricted epitope from chicken ovalbumin (OVA; SIINFEKL, SEQ ID NO:1), or variants with lower affinity (SIINTEKL (SEQ ID NO:2, FT), SIINFEKP (SEQ ID NO:7, LP)) were synthesized, and the purity and identity of each peptide was confirmed by mass spectrometry and gas chromatography (LifeTein, LLC., Hillsborough, N.J.).

In vitro T-cell stimulation: Spleens were collected from OT-1 mice, processed through a mesh screen, and splenocytes were isolated by centrifugation after red blood cell osmotic lysis with ammonium chloride/potassium chloride lysis buffer (0.15 M NH4Cl, 10 mM KHCO3, 0.1 mM EDTA). Splenocytes were cultured at 2×10⁶/mL in RPMI 1640 medium supplemented with L-glutamine, 10% fetal calf serum (FCS), 200 U/mL penicillin/streptomycin, 1% sodium pyruvate, 1% HEPES, 50 μM β-MeOH, and 2 μg/mL of the designated peptide. TLR agonists were all purchased from InvivoGen (San Diego, Calif.) and added one hour before peptides at the following optimal concentrations based on preliminary studies and previously reported results: 300 ng/ml Pam3CSK4, 10 μg/mL Poly(I:C) HMW, 10 μg/ml MPLAs, 3 μg/ml Gardiquimod, 10 μg/ml R848, 5 μM ODN 1826. At the time points indicated, cells were stained, fixed for 15 min at 4° C. in cytofix (BD Biosciences (BD), San Jose, Calif.; 554655), and frozen in FCS+10% DMSO. After all time points were collected, cells from all times were thawed, rinsed and resuspended in PBS+3% FCS+1 mM EDTA and analyzed by flow cytometry. Cells were stained for 30 min at 4° C. in a 1:4 dilution of brilliant stain buffer (BD 563794) in PBS+3% FCS+1mM EDTA. In order to assess co-stimulation, blocking antibodies for CD80 (0.6 μg/mL), CD86 (0.25 μg/mL) or OX40L (0.6 μg/mL) were added 1 hour prior to antigen.

RNA preparation and sequencing: Cells were stimulated in vitro as above, and at the times indicated T cells were isolated via immunomagnetic negative selection (StemCell, Vancouver, Canada 19853), RNA was collected as per the manufacturers instruction (Direct-zol RNA MiniPrep Plus w/TRI Reagent, Zymo Research), treated with DNAse (TURBO DNA-free Kit, Invitrogen), and stored at −80° C. until analysis at the UW-Madison Biotechnology Center. Total RNA was verified for purity and integrity via a NanoDrop2000 Spectrophotometer and Agilent 2100 BioAnalyzer, respectively. Samples that met the Illumina sample input guidelines were prepared using the Illumina® TruSeq® Stranded mRNA Sample Preparation kits (Illumina Inc., San Diego, Calif., USA). For each library preparation, mRNA was purified from 1000 ng total RNA using poly-T oligo-attached magnetic beads. Subsequently, each poly-A enriched sample was fragmented using divalent cations under elevated temperature. The fragmented RNA was synthesized into double-stranded cDNA using SuperScript II Reverse Transcriptase (Invitrogen, Carlsbad, Calif., USA) and random primers for first strand cDNA synthesis followed by second strand synthesis using DNA Polymerase I and RNAse H for removal of mRNA. Double-stranded cDNA was purified by paramagnetic beads (Agencourt AMPure XP beads, Beckman Coulter). The cDNA products were incubated with Klenow DNA Polymerase and DNA fragments, ligated to Illumina adapters, and purified by paramagnetic beads. Adapter ligated DNA was amplified in a Linker Mediated PCR reaction (LM-PCR) for 11 cycles using Phusion™ DNA Polymerase and Illumina's PE genomic DNA primer set and then purified by paramagnetic beads. Quality and quantity of the finished libraries were assessed using an Agilent DNA1000 chip (Agilent Technologies, Inc., Santa Clara, Calif., USA) and Qubit® dsDNA HS Assay Kit (Invitrogen, Carlsbad, Calif., USA), respectively. Libraries were standardized to 2 μM. Cluster generation was performed using standard Cluster Kit (v3) and the Illumina Cluster Station. Single 100 bp sequencing was performed, using standard SBS chemistry (v4) on an Illumina HiSeq2500 sequencer. Images were analyzed using the standard Illumina Pipeline, version 1.8.2.

RNA sequencing data analysis: Illumina sequencing reads were adapter and quality trimmed using the Skewer trimming program. Quality reads were subsequently aligned to the annotated reference genome using the STAR aligner (43). Quantification of expression for each gene was calculated by RSEM (44), the expected read counts from RSEM were filtered for low/empty values and used for differential gene expression analysis using DESeq2 (45). All remaining genes from the pairwise comparisons were analyzed using Gene Set Enrichment Analysis (GSEA) (46, 47) to identify pathways (based on GO terms (c5.all.v6) from the Molecular Signatures Database (MSigDB (48)) as well as custom gene sets involved in TLR1/2 versus TLR7 ligand activation. GSEA was performed by calculating a ranked vector as log2 fold-change. Analysis was performed using R 3.3.1 (49) and Bioconductor 2.32.0 (50). Venn diagrams were generated using the VennDiagram package.

In vitro co-culture experiments: APC subsets were enriched from splenocytes using PE-labeled antibodies specific for either CD19 or CD11c (StemCell). CD8+ T cells were isolated as above. Primary dendritic cells were harvested from spleens of mice previously inoculated with a B16 tumor cell line transduced to secrete FMS like Tyrosine kinase 3 ligand (Flt3-L) as previously described (51). After enrichment, each APC subset, and a subset of T cells with no APC present, were cultured at 2×10⁶ cells/mL in PBS in the presence of 2 μg/mL peptide, with or without TLR agonists as described above, for 1 h. Cells were then rinsed and transferred to fresh medium without peptide or TLR agonist. Naive T cells were added at a 1:1 ratio and incubated for three days, after which cells were stained and analyzed by flow cytometry. To assess the effects of secreted factors as a result of the TLR agonists, cells were incubated as described for 4 hours, rinsed, and incubated for 24 hours in fresh medium. This conditioned medium was then removed and added to a well containing untreated APC and OT-1 T-cells, isolated as above, and incubated for an additional 24 hours before analysis by flow cytometry. Where indicated, 0.1 ng/mL recombinant IL-12 (R&D systems, Minneapolis, Minn., 419-ML-010) was added in place of the TLR agonists or 0.06 μg/mL blocking antibody (R&D systems, AF-419-SP) was added in addition to the agonists. Where indicated, IL-12 concentrations in culture supernatant were detected by ELISA according to the manufacturer's recommendations (R&D systems, kit M1270).

Adoptive transfer and immunization of wild type C57BL/6 (B6) mice: OT-1 splenocytes were harvested and CD8+ T cells were isolated as described above. 2×10⁶ OT-1 T cells were adoptively transferred into 6- to 10-week old female B6 mice via intraperitoneal injection. The day following transfer, mice were immunized subcutaneously with 100 μg SIINFEKL peptide (SEQ ID NO:1) in PBS without adjuvant, intradermally with 100 μg plasmid encoding full length secreted chicken egg ovalbumin (pCI-neo-sOVA (psOVA), Addgene plasmid #25098, Cambridge, Mass.), intraperitoneally with 5×10⁶ cfu of a Listeria monocytogenes vector encoding ovalbumin (Lm-OVA, gift of J D Sauer), or vehicle or corresponding wild-type Lm or DNA plasmid vector. TLR agonists were co-injected with the vaccine at the following concentrations: 20 μg/mouse Pam3CSK4, 100 μg/mouse Gardiquimod, 50 μg/mouse ODN 1826, or vehicle. Mice were euthanized at the times indicated. Spleens were collected, processed as described above, and T-cell populations analyzed by flow cytometry. Data collected on different days were normalized using rainbow beads (Spherotech, Lake Forest, Ill.; RFP-30-5A).

Ovalbumin tumor treatment studies: 10⁶ ovalbumin-expressing E.G7 PD-L1^{high} cells were injected subcutaneously into 6- to10-week old female B6 mice. Ten to fifteen days post injection, when tumors were palpable and similarly sized (˜0.2 cm³), 2×10⁶naïve OT-1 CD8+ T cells were adoptively transferred to each mouse as described above. The following day mice were immunized subcutaneously with 100 μg of an individual peptide (SIINFEKL (SEQ ID NO:1) or variant, LifeTein) in PBS, with or without TLR agonists as described above. In the group receiving PD-1 blocking antibody, 100 μg of antibody (clone G4 (52)) was injected intraperitoneally on the day following vaccination. Tumor volume was measured over time using calipers and calculated in cubic centimeters according to the following formula: (π/6)*(long axis)*(short axis)². Tumors obtained at necropsy were digested in media containing 1 mg/mL collagenase and 20 μg/mL DNAse I (Sigma, St. Louis, Mo.) for 1-2 hours at 37° C., and passed through a 100-μm screen to obtain a single-cell suspension. Tumor cells and tumor-infiltrating lymphocytes (TILs) were stained and analyzed by flow cytometry using the following panel: CD3-FITC (BD 555274), CD4-BUV395 (BD 563790), CD8-BUV805 (BD 564920), LAG-3-BV711 (BD 563179), PD1-PECF594 (BD 562523), PDL1-PECy7 (BD 558017), 41BB-PerCPeF710 (eBioscience 46-1371-82), CD45-BV510 (BD 563891) and Live/Dead Ghost dye 780 (Tonbo, San Diego, Calif. 13-0865-T100) or corresponding fluorescently labeled IgG controls.

SSX2 tumor treatment studies: 6- to 8-week old HHDII-DR1 mice were inoculated with 10⁵ A2/Sarc-SSX2 cells administered subcutaneously, and then immunized intradermally with DNA vaccines weekly beginning one day after tumor implantation, as previously described (42). TLR agonists or vehicle were co-administered with the vaccine intradermally, and tumor volumes were measured over time using calipers, as described above.

Statistical Analyses: Comparison of group means was performed using GraphPad Prism software, v5.01. Analysis of Variance (ANOVA; one- or two-sided depending on the nature of the data) statistical modeling was used to analyze data sets, followed by the Bonferroni multiple-comparison post-hoc procedure to compare individual group means. For all comparisons, P values equal to or less than 0.05 (or 0.01 where indicated) were considered statistically significant.

### REFERENCES FOR EXAMPLE 6

1. Day, C. L., Kaufmann, D. E., Kiepiela, P., Brown, J. A., Moodley, E. S., Reddy, S., Mackey, E. W., Miller, J. D., Leslie, A. J., DePierres, C., et al. 2006. PD-1 expression on HIV-specific T cells is associated with T-cell exhaustion and disease progression. Nature 443:350-354.
2. Tian, X., Zhang, A., Qiu, C., Wang, W., Yang, Y., Qiu, C., Liu, A., Zhu, L., Yuan, S., Hu, H., et al. 2015. The upregulation of LAG-3 on T cells defines a subpopulation with functional exhaustion and correlates with disease progression in HIV-infected subjects. J Immunol 194:3873-3882.
3. Jin, H. T., Anderson, A. C., Tan, W. G., West, E. E., Ha, S. J., Araki, K., Freeman, G. J., Kuchroo, V. K., and Ahmed, R. 2010. Cooperation of Tim-3 and PD-1 in CD8 T-cell exhaustion during chronic viral infection. Proc Natl Acad Sci USA 107:14733-14738.
4. Ferris, R. L., Lu, B., and Kane, L. P. 2014. Too much of a good thing? Tim-3 and TCR signaling in T cell exhaustion. J Immunol 193:1525-1530.
5. Riley, J. L. 2009. PD-1 signaling in primary T cells. Immunol Rev 229:114-125.
6. Sakuishi, K., Apetoh, L., Sullivan, J. M., Blazar, B. R., Kuchroo, V. K., and Anderson, A. C. 2010. Targeting Tim-3 and PD-1 pathways to reverse T cell exhaustion and restore anti-tumor immunity. J Exp Med 207:2187-2194.
7. Haymaker, C., Wu, R., Bematchez, C., and Radvanyi, L. 2012. PD-1 and BTLA and CD8(+) T-cell “exhaustion” in cancer: “Exercising” an alternative viewpoint. Oncoimmunology 1:735-738.
8. Zahm, C. D., Colluru, V. T., and McNeel, D. G. 2017. Vaccination with High-Affinity Epitopes Impairs Antitumor Efficacy by Increasing PD-1 Expression on CD8+ T Cells. Cancer Immunol Res 5:630-641.
9. Rekoske, B. T., Smith, H. A., Olson, B. M., Maricque, B. B., and McNeel, D. G. 2015. PD-1 or PD-L1 Blockade Restores Antitumor Efficacy Following SSX2 Epitope-Modified DNA Vaccine Immunization. Cancer Immunol Res 3:946-955.
10. McNeel, D. G., Eickhoff, J., Jeraj, R., Staab, M. J., Straus, J., Scarpelli, M., Wargowski, E., and Liu, G. 2017. DNA vaccine with pembrolizumab elicits anti-tumor responses in patients with metastatic, castration-resistant prostate cancer (mCRPC). 2017 ASCO-SITC Clinical Immuno-Oncology Symposium (Orlando, Fla.): abstract 168.
11. Takeda, K., and Akira, S. 2004. TLR signaling pathways. Semin Immunol 16:3-9.
12. Nembrini, C., Abel, B., Kopf, M., and Marsland, B. J. 2006. Strong TCR signaling, TLR ligands, and cytokine redundancies ensure robust development of type 1 effector T cells. J Immunol 176:7180-7188.
13. Geng, D., Zheng, L., Srivastava, R., Asprodites, N., Velasco-Gonzalez, C., and Davila, E. 2010. When Toll-like receptor and T-cell receptor signals collide: a mechanism for enhanced CD8 T-cell effector function. Blood 116:3494-3504.
14. Mills, K. H. 2011. TLR-dependent T cell activation in autoimmunity. Nat Rev Immunol 11:807-822.
15. Olino, K., Wada, S., Edil, B. H., Pan, X., Meckel, K., Weber, W., Slansky, J., Tamada, K., Lauer, P., Brockstedt, D., et al. 2012. Tumor-associated antigen expressing Listeria monocytogenes induces effective primary and memory T-cell responses against hepatic colorectal cancer metastases. Ann Surg Oncol 19 Suppl 3:S597-607.
16. Wong, R. M., Smith, K. A., Tam, V. L., Pagarigan, R. R., Meisenburg, B. L., Quach, A. M., Carrillo, M. A., Qiu, Z., and Bot, A. I. 2009. TLR-9 signaling and TCR stimulation co-regulate CD8(+) T cell-associated PD-1 expression. Immunol Lett 127:60-67.
17. Yin, P., Liu, X., Mansfield, A. S., Harrington, S. M., Li, Y., Yan, Y., and Dong, H. 2016. CpG-induced antitumor immunity requires IL-12 in expansion of effector cells and down-regulation of PD-1. Oncotarget 7:70223-70231.
18. Hacker, H. 2000. Signal transduction pathways activated by CpG-DNA. Curr Top Microbiol Immunol 247:77-92.
19. Klinman, D. M. 2006. Adjuvant activity of CpG oligodeoxynucleotides. Int Rev Immunol 25:135-154.
20. Pavlenko, M., Leder, C., Moreno, S., Levitsky, V., and Pisa, P. 2007. Priming of CD8+ T-cell responses after DNA immunization is impaired in TLR9- and MyD88-deficient mice. Vaccine 25:6341-6347.
21. Chu, R. S., Targoni, O. S., Krieg, A. M., Lehmann, P. V., and Harding, C. V. 1997. CpG oligodeoxynucleotides act as adjuvants that switch on T helper 1 (Th1) immunity. J ExpMed 186:1623-1631.
22. Lonsdorf, A. S., Kuekrek, H., Stern, B. V., Boehm, B. O., Lehmann, P. V., and Tary-Lehmann, M. 2003. Intratumor CpG-oligodeoxynucleotide injection induces protective antitumor T cell immunity. J Immunol 171:3941-3946.
23. Wille-Reece, U., Flynn, B. J., Lore, K., Koup, R. A., Miles, A. P., Saul, A., Kedl, R. M., Mattapallil, J. J., Weiss, W. R., Roederer, M., et al. 2006. Toll-like receptor agonists influence the magnitude and quality of memory T cell responses after prime-boost immunization in nonhuman primates. J Exp Med 203:1249-1258.
24. Hartmann, G., Weeratna, R. D., Ballas, Z. K., Payette, P., Blackwell, S., Suparto, I., Rasmussen, W. L., Waldschmidt, M., Sajuthi, D., Purcell, R. H., et al. 2000. Delineation of a CpG phosphorothioate oligodeoxynucleotide for activating primate immune responses in vitro and in vivo. J Immunol 164:1617-1624.
25. Wong, R. M., Smith, K. a., Tam, V. L., Pagarigan, R. R., Meisenburg, B. L., Quach, A. M., Carrillo, M. a., Qiu, Z., and Bot, A. I. 2009. TLR-9 signaling and TCR stimulation co-regulate CD8(+) T cell-associated PD-1 expression. Immunology letters 127:60-67.
26. Rolf, N., Kariminia, A., Ivison, S., Reid, G. S., and Schultz, K. R. 2015. Heterodimer-specific TLR2 stimulation results in divergent functional outcomes in B-cell precursor acute lymphoblastic leukemia. Eur J Immunol 45:1980-1990.
27. Liu, J., Jiang, M., Ma, Z., Dietze, K. K., Zelinskyy, G., Yang, D., Minter, U., Schlaak, J. F., Roggendorf, M., and Lu, M. 2013. TLR1/2 ligand-stimulated mouse liver endothelial cells secrete IL-12 and trigger CD8+ T cell immunity in vitro. J Immunol 191:6178-6190.
28. Halliday, A., Turner, J. D., Guimaraes, A., Bates, P. A., and Taylor, M. J. 2016. The TLR2/6 ligand PAM2CSK4 is a Th2 polarizing adjuvant in Leishmania major and Brugia malayi murine vaccine models. Parasit Vectors 9:96.
29. Jayakumar, A., Castilho, T. M., Park, E., Goldsmith-Pestana, K., Blackwell, J. M., and McMahon-Pratt, D. 2011. TLR1/2 activation during heterologous prime-boost vaccination (DNA-MVA) enhances CD8+ T Cell responses providing protection against Leishmania (Viannia). PLoS Negl Trop Dis 5:e1204.
30. Chodisetti, S. B., Gowthaman, U., Rai, P. K., Vidyarthi, A., Khan, N., and Agrewala, J. N. 2015. Triggering through Toll-like receptor 2 limits chronically stimulated T-helper type 1 cells from undergoing exhaustion. J Infect Dis 211:486-496.
31. Syed, T. A. 2001. A review of the applications of imiquimod: a novel immune response modifier. Expert Opin Pharmacother 2:877-882.
32. Craft, N., Bruhn, K. W., Nguyen, B. D., Prins, R., Lin, J. W., Liau, L. M., and Miller, J. F. 2005. The TLR7 agonist imiquimod enhances the anti-melanoma effects of a recombinant Listeria monocytogenes vaccine. J Immunol 175:1983-1990.
33. Chuang, C. M., Monie, A., Hung, C. F., and Wu, T. C. 2010. Treatment with imiquimod enhances antitumor immunity induced by therapeutic HPV DNA vaccination. J Biomed Sci 17:32.
34. Zahm, C. D., Colluru, V., and McNeel, D. G. 2017. Vaccination with High-Affinity Epitopes Impairs Antitumor Efficacy by Increasing PD-1Expression on CD8+ T Cells. Cancer Immunol Res.
35. Ma, F., Zhang, J., Zhang, J., and Zhang, C. 2010. The TLR7 agonists imiquimod and gardiquimod improve DC-based immunotherapy for melanoma in mice. Cell Mol Immunol 7:381-388.
36. Wang, S., Chen, Z., Hu, C., Qian, F., Cheng, Y., Wu, M., Shi, B., Chen, J., Hu, Y., and Yuan, Z. 2013. Hepatitis B virus surface antigen selectively inhibits TLR2 ligand-induced IL-12 production in monocytes/macrophages by interfering with JNK activation. J Immunol 190:5142-5151.
37. Fischetti, L., Zhong, Z., Pinder, C. L., Tregoning, J. S., and Shattock, R. J. 2017. The synergistic effects of combining TLR ligand based adjuvants on the cytokine response are dependent upon p38/JNK signalling. Cytokine.
38. Ngiow, S. F., Young, A., Blake, S. J., Hill, G. R., Yagita, H., Teng, M. W., Korman, A. J., and Smyth, M. J. 2016. Agonistic CD40 mAb-Driven IL12 Reverses Resistance to Anti-PD1 in a T-cell-Rich Tumor. Cancer Res 76:6266-6277.
39. Gerner, M. Y., Heltemes-Harris, L. M., Fife, B. T., and Mescher, M. F. 2013. Cutting edge: IL-12 and type I IFN differentially program CD8 T cells for programmed death 1 re-expression levels and tumor control. J Immunol 191:1011-1015.
40. Wang, H., Li, J., Han, Q., Yang, F., Xiao, Y., Xiao, M., Xu, Y., Su, L., Cui, N., and Liu, D. 2017. IL-12 Influence mTOR to Modulate CD8+ T Cells Differentiation through T-bet and Eomesodermin in Response to Invasive Pulmonary Aspergillosis. Int J Med Sci 14:977-983.
41. Pajot, A., Michel, M. L., Fazilleau, N., Pancre, V., Auriault, C., Ojcius, D. M., Lemonnier, F. A., and Lone, Y. C. 2004. A mouse model of human adaptive immune functions: HLA-A2.1-/HLA-DR1-transgenic H-2 class I-/class II-knockout mice. Eur J Immunol 34:3060-3069.
42. Rekoske, B. T., Smith, H. A., Olson, B. M., Maricque, B. B., and McNeel, D. G. 2015. PD-1 or PD-L1 Blockade Restores Antitumor Efficacy Following SSX2 Epitope-Modified DNA Vaccine Immunization. Cancer immunology research 3:946-955.
43. Dobin, A., Davis, C. A., Schlesinger, F., Drenkow, J., Zaleski, C., Jha, S., Batut, P., Chaisson, M., and Gingeras, T. R. 2013. STAR: ultrafast universal RNA-seq aligner. Bioinformatics 29:15-21.
44. Li, B., and Dewey, C. N. 2011. RSEM: accurate transcript quantification from RNA-Seq data with or without a reference genome. BMC Bioinformatics 12:323.
45. Love, M. I., Huber, W., and Anders, S. 2014. Moderated estimation of fold change and dispersion for RNA-seq data with DESeq2. Genome Biol 15:550.
46. Mootha, V. K., Lindgren, C. M., Eriksson, K. F., Subramanian, A., Sihag, S., Lehar, J., Puigserver, P., Carlsson, E., Ridderstrale, M., Laurila, E., et al. 2003. PGC-1alpha-responsive genes involved in oxidative phosphorylation are coordinately downregulated in human diabetes. Nat Genet 34:267-273.
47. Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. 2005. Gene set enrichment analysis: a knowledge-based approach for interpreting genome-wide expression profiles. Proc Natl Acad Sci USA 102:15545-15550.
48. Liberzon, A., Birger, C., Thorvaldsdottir, H., Ghandi, M., Mesirov, J. P., and Tamayo, P. 2015. The Molecular Signatures Database (MSigDB) hallmark gene set collection. Cell Syst 1:417-425.
49. R Core Team 2016. R: A Language and Environment for Statistical Computing. R Foundation for Statistical Computing, Vienna, Austria.
50. Huber, W., Carey, V. J., Gentleman, R., Anders, S., Carlson, M., Carvalho, B. S., Bravo, H. C., Davis, S., Gatto, L., Girke, T., et al. 2015. Orchestrating high-throughput genomic analysis with Bioconductor. Nat Methods 12:115-121.
51. Colluru, V. T., McNeel, D. G., Teja Colluru, V., and McNeel, D. G. 2016. B lymphocytes as direct antigen-presenting cells for anti-tumor DNA vaccines. Oncotarget 5.
52. Hirano, F., Kaneko, K., Tamura, H., Dong, H., Wang, S., Ichikawa, M., Rietz, C., Flies, D. B., Lau, J. S., Zhu, G., et al. 2005. Blockade of B7-H1 and PD-1 by monoclonal antibodies potentiates cancer therapeutic immunity. Cancer Res 65:1089-1096.

### Example 7

Determining whether TLR agonist stimulation of T cells similarly affects expression of checkpoint molecules on human CD8+ T cells following activation. The studies above are performed in murine systems. It is known that there are differences in expression of TLR in murine and human cells [46]. In fact, 10 TLRs have been identified in humans, and 12 in mice [47]. Hence it will be important for future human trials combining vaccination with TLR agonist activation to understand if related agents can function similarly in human cells. To test this, we will take advantage of our previous observation that many patients with advanced prostate cancer have detectable CD8+ T cells for the tumor-associated antigen SSX-2 that can be identified by tetramer staining for the HLA-A2-restricted p103 epitope [16]. Using cells from these patients, we have previously identified that stimulating autologous antigen-presenting cells with plasmid DNA encoding SSX2 results in the expansion of p103-specific tetramer+CD8+ T cells that can be detected after 7 days [45]. Further, we have identified that modifications to increase the MHC class I affinity of the p103 epitope, whether delivered as a peptide or included within a DNA vaccine, elicit peptide-specific CD8+ T cells with higher PD-1 expression (FIG. 1) [21]. Consequently, for these studies we take PBMC from human subjects previously identified to have SSX2-specific CD8+ T cells, and culture them in vitro in the presence of plasmid DNA encoding SSX2 or plasmid DNA encoding SSX2 with a modification to encode a higher-affinity p103 epitope. Cultures also include gradients of TLR agonists, including Pam3CSK4 (TLR1/2 agonist) and Resiquimod (TLR7 agonist). After 7 days, cultures are assessed for the frequency of p103 tetramer+ CD8+ T cells, and for expression of checkpoint molecules (PD-1, LAG-3, TIM3, CTLA-4, BTLA, CD160, CD244, VISTA) and markers of activation (4-1BB, CD69). As in the murine studies, cells are evaluated for cytokine/effector function following restimulation with the p103 epitope and using intracellular cytokine staining to evaluate for expression of IFNγ, TNFα, IL-2, IL-4, IL-10, granzyme A, granzyme B, and granulysin.

We have cryopreserved thousands of aliquots of PBMC from subjects with prostate cancer, collected by leukapheresis that will serve as the resources. Studies are performed in multiple replicates with at least three different donors. We anticipate that activating T cells using a plasmid encoding the high-affinity epitope will expand antigen-specific CD8+ T cells with high PD-1 expression, as we observed in murine studies [21]. We expect that TLR1/2 and/or TLR7 activation will lead to expansion of antigen-specific CD8+ T cells with lower PD-1 expression. We further expect that these cells will have functional cytokine/enzyme secreting function with a Th1 bias.

1. A method of eliciting an anti-tumor response in a subject in need thereof, the method comprising administering at least one TLR1/2 agonist or TLR7 agonist and an anti-tumor vaccine to the subject in an effective amount to elicit an anti-tumor response in said subject.

2. The method of claim 1, wherein the anti-tumor response is a cell-mediated response.

3. The method of claim 1, wherein the anti-tumor response is a CD8+ T cell response.

4. The method of claim 2, wherein the CD8+ T cells express low levels or do not express PD-1.

5. The method of claim 4, wherein the CD8+ T cells do not express PD-1.

6. The method of claim 1, wherein the method comprises administering a TLR1/2 agonist in combination with a TLR7 agonist.

7. The method of claim 1, wherein the anti-tumor vaccine is a DNA vaccine.

8. The method of claim 7, wherein the DNA vaccine is specific for prostate cancer.

9. The method of claim 1, wherein the anti-tumor response is to a prostate tumor.

10. A method of increasing the immune response to an antigen, the method comprising administering at least one TLR1/2 agonist or TLR7 agonist and at least one vaccine directed to said antigen in a subject, wherein the immune response to said antigen is increased relative to a subject treated with the vaccine alone.

11. The method of claim 10, wherein the immune response is a cellular immune response.

12. The method of claim 10, wherein the immune response is a CD8+ T cell response.

13. The method of claim 12, wherein the CD8+ T cells exhibit a PD-1^{low} phenotype.

14. The method of claim 10, wherein the antigen is a tumor antigen.

15. The method of claim 10, wherein the antigen is an infectious agent.

16. The method of claim 10, wherein the subject is administered a TLR1/2 agonist in combination with a TLR7 agonist.

17. The method of claim 10, wherein the vaccine is a DNA vaccine.

18. A kit for eliciting an anti-tumor response, the kit comprising:
at least one TLR1/2 agonist or TLR7 agonist; and
at least one immunotherapeutic agent.

19. The kit of claim 18, wherein the immunotherapeutic agent is a vaccine.

20. A composition for eliciting an anti-tumor response, the composition comprising at least one TLR1/2 agonist and/or at least one TLR7 agonist and at least one immunotherapeutic agent.

21. The composition of claim 20, wherein the composition further comprises a pharmaceutically acceptable carrier.


SEQUENCE LISTING (15 sequences)

SEQ ID NO: 1 - protein, 8 aa, Gallus gallus
SIINFEKL

SEQ ID NO: 2 - protein, 8 aa, Artificial Sequence
  synthetic
SIINTEKL

SEQ ID NO: 3 - protein, 9 aa, Homo sapiens
LLLFSIIPV

SEQ ID NO: 4 - protein, 10 aa, Homo sapiens
RMLYFAPDLV

SEQ ID NO: 5 - protein, 9 aa, Homo sapiens
FLCMKALLL

SEQ ID NO: 6 - protein, 9 aa, Homo sapiens
QLTKLLDSV

SEQ ID NO: 7 - protein, 8 aa, Artificial Sequence
  synthetic
SIINFEKP

SEQ ID NO: 8 - protein, 8 aa, Artificial Sequence
  synthetic
SIINFEKY

SEQ ID NO: 9 - DNA, 10070 bp, Homo sapiens
gcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttcaaaaaa

SEQ ID NO: 10 - DNA, 10063 bp, Mus musculus
cagcgccccctcggagatccctaggagccagcctgctgggagaaccagagggtccggagcaaacctggaggctgagagggcatcagaggggaaaagactgagctagccactccagtgccatacagaagcttaagggacgcaccacgccagccccagcccagcgacagccaacgcctgttgcagagcggcggcttcgaagccgccgcccaggagctgccctttcctcttcggtgaagtttctaaaagctgcgggagactcagaggaagcaaggaaagtgtccggtaggactacggctgcctttgtcctcttcccctctacccttaccccctcctgggtcccctctccaggagctgactaggcaggctttctggccaaccctctcccctacacccccagctctgccagccagtttgcacagaggtaaactccctttggctgagagtaggggagcttgttgcacattgcaaggaaggcttttgggagcccagagactgaggagcaacagcacgcccaggagagtccctggttccaggttctcgcccctgcacctcctcctgcccgcccctcaccctgtgtgtggtgttagaaatgaaaagatgaaaaggcagctagggtttcagtagtcgaaagcaaaacaaaagctaaaagaaaacaaaaagaaaatagcccagttcttatttgcacctgcttcagtggactttgaatttggaaggcagaggatttccccttttccctcccgtcaaggtttgagcatcttttaatctgttcttcaagtatttagagacaaactgtgtaagtagcagggcagatcctgtcttgcgcgtgccttcctttactggagactttgaggttatctgggcactccccccacccaccccccctcctgcaagttttcttccccggagcttcccgcaggtgggcagctagctgcagatactacatcatcagtcaggagaactcttcagagcaagagacgaggaggcaggataagggaattcggtggaagctacagacaagctcaaggatggaggtgcagttagggctgggaagggtctacccacggcccccatccaagacctatcgaggagcgttccagaatctgttccagagcgtgcgcgaagcgatccagaacccgggccccaggcaccctgaggccgctaacatagcacctcccggcgcctgtttacagcagaggcaggagactagcccccggcggcggcggcggcagcagcacactgaggatggttctcctcaagcccacatcagaggccccacaggctacctggccctggaggaggaacagcagccttcacagcagcaggcagcctccgagggccaccctgagagcagctgcctccccgagcctggggcggccaccgctcctggcaaggggctgccgcagcagccaccagctcctccagatcaggatgactcagctgccccatccacgttgtccctgctgggccccactttcccaggcttaagcagctgctccgccgacattaaagacattttgaacgaggccggcaccatgcaacttcttcagcagcagcaacaacagcagcagcaccaacagcagcaccaacagcaccaacagcagcaggaggtaatctccgaaggcagcagcgcaagagccagggaggccacgggggctccctcttcctccaaggatagttacctagggggcaattcaaccatatctgacagtgccaaggagttgtgtaaagcagtgtctgtgtccatgggattgggtgtggaagcattggaacatctgagtccaggggaacagcttcggggagactgcatgtacgcgtcgctcctgggaggtccacccgcggtgcgtcccactccttgtgcgccgctgcccgaatgcaaaggtcttcccctggacgaaggcccaggcaaaagcactgaagagactgctgagtattcctctttcaagggaggttacgccaaaggattggaaggtgagagcttggggtgctctggcagcagtgaagcaggtagctctgggacacttgagatcccgtcctctctgtctctgtataaatctggagcactagacgaggcagcagcataccagaatcgcgactactacaactttccgctggctctgtccgggccgccgcaccccccgccccctacccatccacacgcccgtatcaagctggagaacccattggactacggcagcgcctgggctgcggcggcagcgcaatgccgctatggggacttgggtagtctacatggagggagtgtagccgggcccagcactggatcgcccccagccaccacctcttcttcctggcatactctcttcacagctgaagaaggccaattatatgggccaggaggcgggggcggcagcagcagcccaagcgatgccgggcctgtagccccctatggctacactcggccccctcaggggctgacaagccaggagagtgactactctgcctccgaagtgtggtatcctggtggagttgtgaacagagtaccctatcccagtcccaattgtgtcaaaagtgaaatgggaccttggatggagaactactccggaccttatggggacatgcgtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaagggaaacagaagtatctatgtgccagcagaaacgattgtaccattgataaatttcggaggaaaaattgcccatcttgtcgtctccggaaatgttatgaagcagggatgactctgggagctcgtaagctgaagaaacttggaaatctaaaactacaggaggaaggagaaaactccaatgctggcagccccactgaggacccatcccagaagatgactgtatcacacattgaaggctatgaatgtcagcctatctttcttaacgtcctggaagccattgagccaggagtggtgtgtgccggacatgacaacaaccaaccagattcctttgctgccttgttatctagcctcaatgagcttggagagaggcagcttgtgcatgtggtcaagtgggccaaggccttgcctggcttccgcaacttgcatgtggatgaccagatggcggtcattcagtattcctggatgggactgatggtatttgccatgggttggcggtccttcactaatgtcaactccaggatgctctactttgcacctgacttggttttcaatgagtaccgcatgcacaagtctcggatgtacagccagtgtgtgaggatgaggcacctgtctcaagagtttggatggctccaaataaccccccaggaattcctgtgcatgaaagcactgctgctcttcagcattattccagtggatgggctgaaaaatcaaaaattctttgatgaacttcgaatgaactacatcaaggaactcgatcgcatcattgcatgcaaaagaaagaatcccacatcctgctcaaggcgcttctaccagctcaccaagctcctggattctgtgcagcctattgcaagagagctgcatcagttcacttttgacctgctaatcaagtcccatatggtgagcgtggactttcctgaaatgatggcagagatcatctctgtgcaagtgcccaagatcctttctgggaaagtcaagcccatctatttccacacacagtgaagatttggaaaccctaatacccaaaacccaccttgttccctttccagatgtcttctgcctgttatataactctgcactacttctctgcagtgccttgggggaaattcctctactgatgtacagtctgtcgtgaacaggttcctcagttctatttcctgggcttctccttctttttttttcttcttccctccctctttcaccctcccatggcacattttgaatctgctgcgtattgtggctcctgcctttgttttgatttctgttgtatttctttgaatctgtgatgatcctcttgtggcccagtgtcaattgtgcttgtttatagcactgtgctgtgtgccaaccaagcaaatgtttactcaccttatgccatggcaaatttagagagctataagtatctggagaagaaacaaacagagagaataaaaagcaaaaacaaaaccaaaaaataaaaaaaacacaaacaaaaaacaaaaccaacaaacaaaacatgctaggtttgtttcttcgtggtatacaaataaacacataggattcccaaagaagccgacagtgactagaagaaagtaaaaaattacaaatccacgaggagtcactgtttttgttcatcctgtttctctgtgggaaacttcagttgttgttaatggctattgccattaaagagcaggttgaccccaaagctttactgatagggtagagagaaaagaggacaaggagggcagatggataaccattacctccccacagcctttgtccctgagtcctagagtgctcagttgcagtgtagttccttgtactgaaatgtgcttcttgtttgaaaacttgtctgcatgtgaatgcctcttccttccaatccttttctctcttaacctctgcttccaccctcaattgactttcaatagcttttctcagagctttgtactatatgctctctttagccaaaacttggccactttcactgaagttatgtcagtgagaagaaagtggaaaggtctgactctttggaaggctctattcagatttatgttcatatttccatgtgtgagccatagcggagctttgtgactggagtcagaggaaaaggaagtgatggcttagccattctcccattagagatagtgaatgatgatgccatagtgcaatcatcctttcctctgcttttaaaggacctagagaccccatgcagccacattctccctgcacaagtcttcagtgttcagtggccctgaacttcaccaaaatgcatttaagccaaggtggtaaagcttgtacacttctttggacgtgtttgtagacactgctaagatctccctctcaccaccaccacaaaggctagcaggccagcagccacagcatctatgtttagatgttaatagcataaaagacatctcactcaatgtctttcatcaacagtaaatttctggagcccttagaaaaattggaaagaaagcatcaaagggaccagacaaaatgggcatcttgcccttgtcctccagagacaatatattcctcccaagtggagaaatgtcaatttcctcctcagaacaattaaaggggctacccagaccatggtggaagagaaaactaagtaacccagctgagaaaaatgaagacactagaaccagaaagcacaggactttttcctttccatccagcatacccattggcagaaataatggaaggaaaagagaaggccagaagaaaatacagactgctgaagtcttcagaggcaaagtctaaagccagatgaataccatctggctagatgggcatcagtttgctcatcctcctctattgccattgctgggctgactttggccaaagttacttcgaatctccaccatagttgtcccctctcagtcagagggtgcaggaccactgaaacattctatccaccgtgactctcattggacagatctggccggtgtggctacaaatagactgcacccataaactcagggcaagccctgggtcactggtttcatgtagtctgttgacagccttctttactgtggactctgttcctcaaccttgagtgcaggaggaatgcacatctacttttgcctttgtatcattcctcctcactcagctcttcacctccctgcagaccttaagaaatcaggggccagctgccaagctgactcttttggttggtactatgttaactgaaaaggtgatttccgaaggacaggttttcttccctgatttctttgttgctattaatagcaaaaacaaacttgcaaaacaacttctttaacaaggaagggaggatatatacaatgggtgatatggtaatccaaccctgcttgacaaaaactgaagctgacaggttacatttaaaaacaaaacaaaacaaaacgggacagtttctgatttgctttgtgacaacaccatctggcttatgtacaggagctctcttagctgttccttaaacagaaaaaaaatcattactccttttagttaaatttggttacattttaatagtttctttacatctattctgaagcaatttttgtcttctgtggtacatggattttattataacattctaatatttgtctttgtaaatactagagactctttgatccatttctctaggaagtttttcatcttatggagttctgaatcatgacttttatctttatgaatgtatatgctttttacttgcaaaagccaaaaagagtgaaacagcagtgcaattaaagcaacaccaactaaactccaaatttccaagtgacaatattagagaaaaacagcatacacatggctttatgcctactgcttctgcggtggggtttgggtgcgcaatggaaactgtagcttggctgtgttctcccacacaagtgaagaagagattggtttttgcttttttggattttgtgtttcttttctgttttgttttgttttgttttgttttgctttgctttctttggccatcaatgttccaactaatatgattggcggagcacgtgctctgctcagtagagtgaatgttgctggtgcactatgctcacctgtgaacggctggccatttctccattcatatggttaagatggaagatgaggatcacttaccagagaagtcaaggtgatcatctccaaagaggtttacagtgcttggtaggaatggaaaatgaggacaagaaaaagaggagaaccatggagaaggcccaactgggcaggacagcagccagctgccaaagtcacgaactctgggattcaagaagagtcgtgtagtgctttcaactctcatccgcaggcagctcactgtgtgtggactctgagctgacacgggagttggcttctttgttccatagattttctatgccacaggcaatattattgttcttggaaagttcattatttttttaaattaccttactctcagaaagggatttttttgaaggattctgtcatatatctttggaaaacagaaaatcagtaatatgtatatttttatgtatgttcactggcactaaaaaaaaaaaaaaaaagaaaagaaaaaaaagagaaaaaaaaaagcttcactctgtcctttgggtagttgctgaggttaattgtccaggttgagaaatgtgcttctgctaacatccttctctgtccacactctatttctaagtacatataggcatatataggaagatatattcaacacactttaagaaaaaagtatgtccaccatccacatgataaccacaatgatactccacaaattacatgactttaagcttcaagcaacttctaactgattcattcatttatagccttgccctcttctttcccttaaatttggcccagcacaaagacccaagccacccttatacctccctaagacttaagccagcaccagacttcagaaggttttctgaagacaactgacttgctatccctgcatgaccctagcatggtcctgcaaacacaagagactaattataattctcctccactaattgcctgggtcacaggtcattgggccaaggccatgattcttatgcttacgaaccactaatgctaacctactagattaaatcctgaactgaaagttaaaagaagccatttagcatgtgaaacttcttggagtaagaagtttctgtcccggctgcctttgcaaacaggtttgctttcaccacttatctccttgaaaatctttgaaggccttttttttttaagtagaaaaggagatgaaagcattatattatgtaaccaaagattatattgtatctaagataccaaattttttaagggcagggaaggagcaagcattagtgcctctttggtaaattatccaaagacagactgaaggacttttctgatgattgacttagaagactttgtggggaggggttgtctcacaatatacatatttagaagtgttgagaataatttggggggaaatgggattatagtgtccttcactaactgattttataagcagaactagctttccttttttttttttttaaagtagttacaaagcaaattcttaaagctccatctttgcatggttagaaatggagctggtcttggccactgtgtttactagtgcccatgttagcttatttgaagatgtgaagcccttgataagaaggggtacatttaaaggattagatttttgcactagaaggagggcaggcagaaaccctcatttctgcccagtttggacagcacaaaaagttctctgcagtttaaggcagaaagttgaaatatattgtaaatgagtatttgtatccatgtttcaaaactgaattctatatatagatgtaatgtgttctgatagctttacctttctctgcacctttatatttggttccaggtcatatctgatgccatgtacttgtaagagaggttgcagttacatttttggatgctctctcagaatggataagacacctggattgatcagataactgagatctcttcccttcttgggcctggtgttgaggccttgcaaaggggtggaagaggaaagggtagggtacatgatgtattgcactttactagcttaagacggatgaatgtggaaagggtggtgaaatttcattgaaaatgcctaggaattgcaatagggagaaatccagatgtggggccaggtgcccacccaaaggactggccagcagcctcttcatgggatctgaggcattgggaaaaggaaggctatttccttggttttcaccatccttgttagagaagggcagttgcctggtcttgggaacctggagcaaacgctccttctgtcacatcaattctttcccctgcaattgaggtgctcttgctactgggtgtccgtgtgctctaattctggttctggatatgttctgtaaagattttgataattgctaatgtatttttctctgttaaaaatttgttagtgtgttagaagtcatatctctgtaggtacagatcctttgctacccatgagtagagggattttttttcttcaattaagagtttgaccctggggtctgttgcccagagcccatccagaaaaaaaaatccacatttgtcacaatttttctgaaatttcagtcaaggtaacagatcgctgggagttctctttacccccccaaaaaagcagataattgaatttagcaggtggtgttttagagcaaaaaacaaaacagcctttgacccagctttaatatgacccaatttaatctggccaggaagcaggtaatgtgtattaattggcttccaatcctggttgagtgtagcaaggttctactttgtttcctagttccttttgttacatggcctttcacagaaaggattgactgggtttgcagtatatcttatggccttagcacctattgctaactgtcctgaagggaattgcctatggggttgtcctataagccacttctatcattaaaagcagccaccaatggaatctcccaggtttgaaaaaaaaaaaaaaaaacagatggtcctttaccattcattgacacacatccctgctttcctgtagacagattgactggacattgattagggaatacatggcaaatgacatgcttacactaccctggagattaatttggcagtaggagggaatagacaatgtaaccaagaatgtaatgtaattcttatagagataagaattaaatctggatgtggagagagcaaagagagaaagcattcaatttttttttcaaaagaaaccaatttattttgcttgaaacttctttcgctggggcttcagttctcacagcggctcttggtctccactgggcagcaggaccagccccaagcgctagtgttctgttctctttttgtaatcttggaatcttttgttgctctaaatacaattaaaaatggcagaaacttgtttgttggaatac

SEQ ID NO: 11 - DNA, 4137 bp, Rattus norvegicus
atccctaggagccagcctgctgggagaaccagagggtccggagcaaacctggaggctgagagggcatcagaggggaaaagactgagttagccactccagtgccatacagaagcttaagggacataccacgccagccccagcccagcgacagccaacgcctgttgcagagcggcggcttcgaagccgccgcccagaagctgccctttcctcttcggtgaagtttctaaaagctgcgggagactcggaggaagcgaagaaagtgtccggtaggactacgactgcctttgtcctcctccctcctacccctacccctcctgggtcccctctccctgagcggactaggcaggcttcctggccagccctctcccctacaccaccagctctgccagccagtttgcacagaggtaactccctttggctgaaagcagacgagcttgttgcccattggaagggaggcttttgggagcccagagactgaggagcaacagcacgctggagagtccctgattccaggttctcccccctgcacctcctactgcccgcccctcaccctgtgtgtgcagctagaattgaaaagatgaaaagacagttggggcttcagtagtcgaaagcaaaacaaaagcaaaaagaaaacaaaaagaaaatagcccagttcttatttgcacctgcttcagtggacattgactttggaaggcagagaattttccttccccccagtcaagctttgagcatcttttaatctgttcttcaagtatttagggacaaactgtgaaactagcagggcagatcctgtctagcgcgtgccttcctttacaggagactttgaggctatctgggcgctcccccccctccctgcaagttttcttccctggagcttcccgcaggtgggcagctagctgcagatactacatcatcagtcagtagaactcttcagagcaagagacgaggaggcaggataagggaattcggtggaagctagagacaagctaaaggatggaggtgcagttagggctgggaagggtctacccacggcccccgtccaagacctatcgaggagcgttccagaatctgttccagagcgtgcgcgaagcgatccagaacccgggccccaggcaccctgaggccgctagcatagcacctcccggtgcctgtttacagcagcggcaggagactagcccccggcggcggcggcggcagcagcaccctgaggatggctctcctcaagcccacatcagaggcaccacaggctacctggccctggaggaggaacagcagccttcacagcagcagtcagcctccgagggccaccctgagagcggctgcctcccggagcctggagctgccacggctcctggcaaggggctgccgcagcagccaccagctcctccagatcaggatgactcagctgccccatccacgttgtccctactgggccccactttcccaggcttaagcagctgctccgcagacattaaagacatcctgagcgaggccggcaccatgcaacttcttcagcagcagcagcaacagcaacagcagcagcagcagcagcagcagcagcagcagcaacagcagcaggaggtaatatccgaaggcagcagcagcgtgagagcaagggaggccactggggctccctcttcctccaaggatagttacctagggggcaattcgaccatatctgacagtgccaaggagttgtgtaaagcagtgtctgtgtccatggggttgggtgtggaagcactggaacatctgagtccaggggagcagcttcggggcgactgcatgtacgcgtcgctcctgggaggtccacccgccgtgcgtcccactccttgtgcgcctctggccgaatgcaaaggtctttccctggacgaaggcccgggcaaaggcactgaagagactgctgagtattcctctttcaagggaggttacgccaaagggttggaaggtgagagtctgggctgctctggcagcagtgaagcaggtagctctgggacacttgagatcccgtcctcactgtctctgtataagtctggagcagtagacgaggcagcagcataccagaatcgcgactactacaactttccgctcgctctgtccgggccgccgcaccccccgccccctacccatccacacgcccgcatcaagctggagaacccgtcggactacggcagcgcctgggctgcggcggcagcgcaatgccgctatggggacttggctagcctacatggagggagtgtagccggacccagcactggatcgcccccagccaccgcctcttcttcctggcatactctcttcacagctgaagaaggccaattatatgggccaggaggcgggggcggcagcagtagcccaagcgatgctgggcctgtagccccctatggctacactcggccccctcaggggctggcaagccaggagggtgacttctctgcctctgaagtgtggtatcctggtggagttgtgaacagagtcccctatcccagtcccagttgtgttaaaagtgaaatgggaccttggatggagaactactccggaccttatggggacatgcgtttggacagtaccagggaccacgttttacccatcgactattacttcccaccccagaagacctgcctgatctgtggagatgaagcttctggttgtcactacggagctctcacttgtggcagctgcaaggtcttcttcaaaagagctgcggaagggaaacagaagtatctatgtgccagcagaaatgattgcaccattgataaatttcggaggaaaaattgtccatcgtgtcgtctccggaaatgttatgaagcagggatgactctgggagctcgtaagctgaagaaacttggaaatctcaaactacaggaagaaggagaaaactccagtgctggtagccccactgaggacccatcccagaagatgactgtatcacacattgaaggctatgaatgtcaacctatctttcttaatgtcctggaagccattgagccaggagtggtgtgtgccggacatgacaacaaccagcctgattcctttgctgccttgttatctagtctcaacgagcttggcgagagacagcttgtacatgtggtcaagtgggccaaggccttgcctggcttccgcaacttgcatgtggatgaccagatggcagtcattcagtattcctggatgggactgatggtatttgccatgggttggcggtccttcactaatgtcaactctaggatgctctactttgcacctgacctggttttcaatgagtatcgcatgcacaagtctcgaatgtacagccagtgcgtgaggatgaggcacctttctcaagagtttggatggctccagataaccccccaggaattcctgtgcatgaaagcactgctactcttcagcattattccagtggatgggctgaaaaatcaaaaattctttgatgaacttcgaatgaactacatcaaggaacttgatcgcatcattgcatgcaaaagaaaaaatcccacatcctgctcaaggcgcttctaccagctcaccaagctcctggattctgtgcagcctattgcaagagagctgcatcaattcacttttgacctgctaatcaagtcccatatggtgagcgtggactttcctgaaatgatggcagagatcatctctgtgcaagtgcccaagatcctttctgggaaagtcaagcccatctatttccacacacagtgaagatttggaaaccctaatacccaaacccaccttgttcccttttcagatgtcttctgcctgttatataactctgcactacttctctggcatgggccttgggggaaattcctctactgatgtacagtctgtcatgaacatgttccccaagttctatttcctgggcttttccttctttctttttcttcttctctgcctcttttaccctcccatggcacattttgaatccgctgcgtgttgtggctcctgcctgtgttttgagttttgttgtatttcttcaagtctgtgatgatcttcttgtggcccagtgtcaactgtgcttgtttatagcactgtgctgtgtgccaaccaagcaaatgtttactcaccttatgccatggcaagtttagagagctataagtatcttgggaagaaacaaacagagagagtaaaaaaacc

SEQ ID NO: 12 - DNA, 3577 bp, Canis lupus
atggaggtacagttagggctagggagggtctacccccggccgccgtccaagacctatcgaggagctttccagaacctgttccagagtgtgcgcgaagtgatccagaacccgggccccaggcaccctgaggccgtgagcgcagcacctcccggtgcccatttgcagcagcagcagcagcagcagcagcagcaggagaccagtcctcggcagcagcagcagcaacagcagggtgacgatggctctccccaagcgcagagcagaggccccacaggctacctggctctggatgaggaacagcagccttcccaacagcggtcagcctccaagggccatccggagagtgcctgcgttccagagcctggagtgacttcggccaccggcaaggggctgcagcagcagcagccagcaccaccggacgagaatgactcagctgccccatccacattgtcactgctgggccccactttcccgggcttaagtagctgttccaccgatcttaaagacatcctgagcgaggctggaaccatgcaactccttcagcagcagcggcagcagcagcagcagcagcagcaacagcaacagcagcagcagcagcagcaacagcaggaggtagtatcagaaggtagcagcagcgggagagcaagggaggccgccggtgcttccacctcctccaaggacagttacctaggtggcagttcgaccatctcggacagcgccaaggagttgtgtaaggcagtgtcggtgtccatgggtttgggtgtggaggcattggaacatctgagccctggggaacagcttcggggggattgtatgtacgccccgctcctgggaggtccacccgccgtacgtccttgcgctccgctggccgaatgcaaaggttctctgctggatgacggcccgggcaagggcaccgaagaaactgctgaatattcccctttcaaggcaggttatgcgaaagggttggatggggacagcctgggctgttcgagcagcagtgaagcagggggctccggaacacttgagatgccatccaccctgtctctttacaagtctggagcactagatgaagcggcagcttatcaaagtcgagactactacaactttcctctctccctaggcgggccgcctccccatccaccacctccccatcctcacacccgcatcaagctggaaaaccctctggactatggcagcgcctgggcggctgcagcggcacaatgccgctacggggatctggcgagcctgcacggagcgggtgcagcaggacccagctcgggctcaccttcggccaccacctcctcttcctggcacactctcttcacagcagaagaaggccagctgtatgggccctgcggcgggagtggaggcggcagtgcaggcgacgggggatctgtagccccctatggctacactcggccacctcagggattggcgggtcaggaaggtgacttccctccacctgatgtgtggtatccgggcggtgtggtgagcagagtgccctttccaagtcctagttgtgtcaaaagcgagatgggctcttggatggagagctactccggaccctatggggacatgcgtttggagactgccagggaccatgttctacccattgactattactttccacctcagaagacctgtctgatctgcggtgatgaagcttctggctgtcactatggagctctcacttgtggaagctgcaaagtcttctttaaaagagccgctgaagggaaacagaagtacctgtgtgccagcagaaatgattgtaccatcgataaattccgaaggaaaaattgtccatcttgtcgcctccggaaatgctatgaagcagggatgactctgggagcccggaagctaaagaaactggggaatctgaaactgcaagaggaaggagaggcttccaatgtcaccagccccactgaggagccaacccagaagctgacggtgtcacacattgaaggctatgagtgtcagcccatctttctgaatgtccttgaagccatcgagccaggcgtggtgtgtgctggacatgacaacaaccagcccgactcctttgcagccttgctctctagccttaatgaattgggtgaaaggcagcttgtacatgtggtcaagtgggccaaggccttgccgggcttccgcaacctgcacgtggatgaccagatggcagtcattcagtactcctggatggggctcatggtgtttgccatgggctggcgatccttcaccaatgtcaactccaggatgctctacttcgcccctgacctggttttcaatgagtaccgcatgcacaagtcccggatgtacagccagtgtgtccgaatgaggcacctctctcaagaatttggatggctccaaatcaccccgcaggaatttttgtgcatgaaggcgctgctgctattcagcattattccagtggatgggctgaaaaatcaaaaattctttgatgaacttcgaatgaactacatcaaggaacttgatcgtatcattgcttgcaagagaaaaaatcccacatcctgctcaaggcgcttctaccagctcaccaagctcctggactctgtgcaacctattgctcgagagctgcatcagttcacttttgacctgctaatcaagtcccacatggtgagcgtggactttccagaaatgatggcagaaatcatctccgtgcaagtgcccaagattctttctgggaaagtcaagcccatctatttccacacgcagtgaagctttagaagcccctgtcttctcaccaccaccaccaccacccccgccccctactttttaaaaccttggtgtttgtttcatctgcttttagaacgtgcgatatttccagtacctacttttttgctgaatccaaagatatataaatataaatatatatattttataaagatcaaaatgatataaaggagatacgtttcttcctttaaaaaaaaacaaatagaaattcattgttaatgttcgtattatgatgccacttttctaaatcgtgcatcttgtatatgtgctgccgaagtgagcactaaatcgtgcatcttgatggaaaggtgtaaatatcaatcaccaggactgcttagttaattggtatttaatatctaggtgaattgggggtatggggtgggggtatgacaggacatacttggcttgccctcctcgaaatgactgccagttttccatatgcctgtccagttccaggaattttgttcacaagtgtgggggcgtcataggagtaaagcctgtaggacagatgaaaccccctgcttcagaggagcaagagctccaaggccaactgtttaacaagcaaggtgtttttttttctttctttctttctctttccaatgtgtggaattttgatgatggaaatgtcattctgagagcaatatttaaaattctcaggaattgacttatagtattgagaatgaattcagttgcaatcaagtttacattaaatgttgcttttaaaaaatctcagtcatacttcacactgcaaagaatccagatcacatgggataacttatgagatggttgagtggtaagctctgactgctttgctgatcattttgtaaataaaggtttc

SEQ ID NO: 13 - DNA, 2869 bp, Pan troglodytes
atggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgcagcagcagcagcagcagcagcagcagcagcagcagcagcagcagcagcagcagcagcagcagcaagagactagccccaggcagcagcagcagcagggtgaggatggttctccccaagcccatcgtagaggccccacaggctacctggtcctggatgaggaacagcaaccttcacagccgcagtcggccccggagtgccaccccgagagaggttgcgtcccagagcctggggccgccgtggccgccagcaaggggctgccgcagcagctgccagcacctccggacgaggatgactcagctgccccatccacgttgtccctgctgggccccactttccccggcttaagcagctgctccgccgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacagcgccaaggagttgtgtaaggcagtgtcggtgtccatgggcctgggtgtggaggcgttggagcatctgagtccgggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtccaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggggggggtggtggcggcggcggcggcggcggcggcggcggcggcgaggcgggagctgtagccccctacggctacactcggccccctcaggggctggcgggccaggaaggcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaacccctatttccccaccccagctcatgccccctttcagatgtcttctgcctgttataactctgcactactcctctgcagtgccttggggaatttcctctattgatgtacagtctgtcatgaac

SEQ ID NO: 14 - DNA, 2821 bp, Macaca fascicularis
atggaggtgcagttagggctggggagggtctaccctcggccgccgtccaagacctaccgaggagctttccagaatctgttccagagcgtgcgcgaagtgatccagaacccgggccccaggcacccagaggccgcgagcgcagcacctcccggcgccagtttgcagcagcagcagcagcagcagcaagaaactagcccccggcaacagcagcagcagcagcagggtgaggatggttctccccaagcccatcgtagaggccccacaggctacctggtcctggatgaggaacagcagccttcacagcctcagtcagccccggagtgccaccccgagagaggttgcgtcccagagcctggagccgccgtggccgccggcaaggggctgccgcagcagctgccagcacctccggacgaggatgactcagctgccccatccacgttgtctctgctgggccccactttccccggcttaagcagctgctccaccgaccttaaagacatcctgagcgaggccagcaccatgcaactccttcagcaacagcagcaggaagcagtatccgaaggcagcagcagcgggagagccagggaggcctcgggggctcccacttcctccaaggacaattacttagggggcacttcgaccatttctgacagcgccaaggagctgtgtaaggcagtgtcggtgtccatgggcttgggtgtggaggcgttggagcatctgagtccaggggaacagcttcggggggattgcatgtacgccccagttttgggagttccacccgctgtgcgtcccactccgtgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtccaccctgtctctctacaagtccggagcactggacgaggcagctgcgtaccagagtcgcgactactacaactttccactggctctggccgggccgccgccccctccaccgcctccccatccccacgctcgcatcaagctggagaacccgctggactatggcagcgcctgggcggctgcggcggcgcagtgccgctatggggacctggcgagcctgcatggcgcgggtgcagcgggacccggctctgggtcaccctcagcggccgcttcctcatcctggcacactctcttcacagccgaagaaggccagttgtatggaccgtgtggtggtgggggcggcggcggtggcggcggcggcggcggcgcaggcgaggcgggagctgtagccccctacggctacactcggccacctcaggggctggcgggccaggaaggcgacttcaccgcacctgatgtgtggtaccctggcggcatggtgagcagagtgccctatcccagtcccacttgtgtcaaaagcgagatgggcccctggatggatagctactccggaccttacggggacatgcggttggagactgccagggaccatgttttgccaattgactattactttccaccccagaagacctgcctgatctgtggagatgaagcttctgggtgtcactatggagctctcacatgtggaagctgcaaggtcttcttcaaaagagccgctgaagggaaacagaagtacctgtgtgccagcagaaatgattgcactattgataaattccgaaggaaaaattgtccatcttgccgtcttcggaaatgttatgaagcagggatgactctgggagcccggaagctgaagaaacttggtaatctgaaactacaggaggaaggagaggcttccagcaccaccagccccactgaggagacagcccagaagctgacagtgtcacacattgaaggctatgaatgtcagcccatctttctgaatgtcctggaagccattgagccaggtgtggtgtgtgctggacatgacaacaaccagcccgactccttcgcagccttgctctctagcctcaatgaactgggagagagacagcttgtacatgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctactttgcccctgatctggttttcaatgagtaccgcatgcacaagtcccggatgtacagccagtgtgtccgaatgaggcacctctctcaagagtttggatggctccaaatcaccccccaggaattcctgtgcatgaaagcgctgctactcttcagcattattccagtggatgggctgaaaaatcaaaaattctttgatgaacttcgaatgaactacatcaaggaactcgatcgtatcattgcatgcaaaagaaaaaatcccacatcctgctcaaggcgtttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaaaatcctttctgggaaagtcaagcccatctatttccacacccagtgaagcattggaaatccctatttcctcaccccagctcatgccccctttcagatgtcttctgcctgttataactctgcactactcctctgcagtgccttggggaatttcctctattgatgtacagtctgtcatgaac

SEQ ID NO: 15 - DNA, 2799 bp, Eulemur fulvus
atggaggtgcagttagggctggggagggtctacccccggccaccgtccaagacctatcgaggagctttccagaatctgttccaaagcgtgcgagaagtgatccagaacccgggccccaggcaccctgaggccgcgagcgcggcacctcctggcgctcgtttgcagcagcagcaggaaaccagccccccgcagcagcagcagcagcagcagggtgaggatggctctccccaagcccagagcagaggccccacaggctacctggccctggacgaggaacagcagccttcacaacagcagtcagccctggagtgccaccctgagagcggctgcgtcccagagcctggagccgccgccgcggccagcaaggggctgcagcagcagccgccagcaccttcggacgaggatgactcagctgtcccatccacgttgtccctgctgggccccactttcccgggcttaagcagctgctcagccgaccttaaagacatcctgagcgaggccggcaccatgcaactacttcagcaacagcagcaggaggcagtatccgaaggcagcagcagcgggagagcgagggaggccgctggggctcccacctcctccaaggacagttacctagggggcacttcgaccatatctgacagcgccaaggagttgtgtaaggcagtgtcggtgtccatgggcttgggtgtggagacgctggagcatctgagtcctggagaacagcttcggggggattgcatgtacgccccgctcttgggaggtccacccgctgtgcgtcccactccctgtgccccactggctgaatgcaaaggttctttgctggatgacagtgcagacaagggcactgaagagcctgctgagtatacccctttcaagggaagttacacccaagggctagaaggcgagagcctgggctgctctggaagcagcgaagcagggagctccgggacacttgagctgccctccactctgtctctctacaagtccggagcactggaggaggccgcatcgtaccagagtcgtgactactacaactttccgctggccctggcagggccaccaccccctccgcttcctcctcatccccacgctcgtatcaagctggagaacccgctggactacggcagctcctgggcggccgcggcagcgcagtgccgctttggggacctggctagcctacacggcgggggtgcaacgggacccggctccggctcgccctcagccgcagccgcctcttcctggcacactctcttcacagccgaagaaggccagttgtatgggccgtgtggtgggggtggcggtggcacaagcgaagctggggccgtaaccccctatggctactctcgaccacctcaggggctggcgggccaggaaggcgacttccctgcacccgatgtgtggtaccccagcggcgtggtgagcagagtgccctatcccagccccagctgtgtcaaaagtgagatgggcccctggatggagagctactctggaccttacggggacgtgcgtttggagactgccagggaccatgttttgcccattgactattactttccaccccagaagacctgccttatctgtggggatgaagcttctggctgtcactatggagctctcacttgtggaagctgcaaggtcttcttcaaaagagctgctgaaggcaaacagaagtacctatgtgccagcagaaatgattgcaccattgataaattccgaaggaaaaattgtccatcttgtcgtctccggaaatgctatgaagcaggaatgactctgggagcccggaagctgaagaaacttggtaatctgaagctacaggaagaaggggaggcttccagcgccaccagtcccactgaggagtccagccagaagctgacggtgtcacacattgaaggctatgaatgtcagcccatctttctgaatgtcctggaagccattgagccaggcgtggtgtgtgctggacatgacaacaaccagcctgactcttttgcagccttgctctctagcctcaatgaactgggcgagagacagcttgttcatgtggtcaagtgggccaaggccttgcctggattccgcaacttgcatgtggacgatcagatggcagtcattcaatattcatggatggggctcatggtgtttgccatgggctggcgatccttcaccaacgtcaactctaggatgctctattttgctcctgatctggttttcaatgagtaccgcatgcacaagtcccggatgtacagccagtgtgtccgaatgaggcacctctctcaagagtttggatggctccaaatcacaccccaggaattcctgtgcatgaaagcactactgcttttcagcattattccagtggatgggctgaaaaatcaaaaattctttgatgaacttcgaatgaactacatcaaggaactcgatcgtatcattgcatgcaaaagaaaaaatcccacatcctgttcaaggcgcttctaccagctcaccaagctcctggactctgtgcagcctattgcaagagagctgcatcagttcacttttgacctgctaatcaagtcccacatggtgagcgtggacttcccggaaatgatggcagagatcatctctgtgcaagtgcccaagatcctttctgggaaagtcaagcccatctatttccacacacagtgaagcattggaatcccccatttctcctcccaaccccactccccttttcagatgtcttctgcctgttataactctgcactactcctctgcagtgccttggggaatttcctctattgatgtacagtctgtcatgaacatgtcccaaaa